SEQUENCE LISTING (21 sequences)

SEQ ID NO: 1 - protein, 32 aa, synthetic construct
  VARIANT 1: note = K or T
TKPCQSDKDCKKFACRKPKVPKCINGFCKCVR

SEQ ID NO: 2 - protein, 33 aa, synthetic construct
ATKPCQSDKDCKKFACRKPKVPKCINGFCKCVR

SEQ ID NO: 3 - protein, 34 aa, synthetic construct
KKMPCKRRRDCKTYPCPHPKVRDCVKGYCKCVVR

SEQ ID NO: 4 - protein, 33 aa, synthetic construct
TKQPCKSRKHCKTYRCPTPKVPNCVNGFCKCVR

SEQ ID NO: 5 - protein, 26 aa, synthetic construct
SRDCKTYPCPPSKVKDCIKGYCKCVR

SEQ ID NO: 6 - protein, 123 aa, synthetic construct
CTCGAGAAAAGAGCTACTAAGCCATGTCAATCTGATAAGGATTGTAAGAAATTTGCTTGTAGAAAGCCAAAAGTTCCTAAGTGTATTAACGGTTTCTGTAAATGTGTTAGATAGTAATCTAGA

SEQ ID NO: 7 - protein, 33 aa, synthetic construct
  VARIANT 1: note = K or T
  VARIANT 3: note = absent, M or Q
  VARIANT 6: note = K or Q
  VARIANT 7: note = R or S
  VARIANT 8: note = R or D
  VARIANT 9: note = R, K, or D
  VARIANT 12: note = T or K
  VARIANT 13: note = Y or F
  VARIANT 14: note = P, A, or R
  VARIANT 16: note = P or R
  VARIANT 17: note = H, K, P, or T
  VARIANT 18: note = P or S
  VARIANT 21: note = R, K, or P
  VARIANT 22: note = D, K, or N
  VARIANT 24: note = I or V
  VARIANT 25: note = K or N
  VARIANT 27: note = F or Y
  VARIANT 32: note = V or absent
BKXPCBBBXCKBBXCBXBKVXXCBBGBCKCVBR

SEQ ID NO: 8 - protein, 8 aa, synthetic construct
GYCKCVVR

SEQ ID NO: 9 - protein, 7 aa, synthetic construct
GFCKCVR

SEQ ID NO: 10 - protein, 7 aa, synthetic construct
GYCKCVR

SEQ ID NO: 11 - protein, 8 aa, synthetic construct
  VARIANT 2: note = F or Y
  VARIANT 7: note = absent or V
GBCKCVBR

SEQ ID NO: 12 - protein, 15 aa, synthetic construct
  VARIANT 6: note = R, K, or P
  VARIANT 4: note = D, K, or N
  VARIANT 3: note = I or V
  VARIANT 7: note = K or N
  VARIANT 9: note = F or Y
  VARIANT 14: note = V or absent
KVBXCXXGBCKCVBR

SEQ ID NO: 13 - protein, 14 aa, synthetic construct
KVPKCINGFCKCVR

SEQ ID NO: 14 - protein, 15 aa, synthetic construct
KVRDCVKGYCKCVVR

SEQ ID NO: 15 - protein, 14 aa, synthetic construct
KVPNCVNGFCKCVR

SEQ ID NO: 16 - protein, 14 aa, synthetic construct
KVKDCIKGYCKCVR

SEQ ID NO: 17 - protein, 6 aa, synthetic construct
KREAEA

SEQ ID NO: 18 - protein, 32 aa, synthetic construct
  DISULFID 10: note = 10...28
  DISULFID 15: note = 15...30
  DISULFID 28: note = 28...10
  DISULFID 30: note = 30...15
TKPSQSDKDCKKFACRKPKVPKSINGFCKCVR

SEQ ID NO: 19 - protein, 32 aa, synthetic construct
  DISULFID 4: note = 4...23
  DISULFID 15: note = 15...30
  DISULFID 23: note = 23...4
  DISULFID 30: note = 30...15
TKPCQSDKDSKKFACRKPKVPKCINGFSKCVR

SEQ ID NO: 20 - protein, 33 aa, synthetic construct
  VARIANT 1: note = K or T
  VARIANT 3: note = absent, M, or Q
  VARIANT 6: note = K or Q
  VARIANT 7: note = R or S
  VARIANT 8: note = R or D
  VARIANT 9: note = R, K, or D
  VARIANT 12: note = T or K
  VARIANT 13: note = Y or F
  VARIANT 14: note = P, A, or R
  VARIANT 16: note = P or R
  VARIANT 17: note = H, K, P, or T
  VARIANT 18: note = P or S
  VARIANT 21: note = R, K, or P
  VARIANT 22: note = D, K, or N
  VARIANT 24: note = I or V
  VARIANT 25: note = K or N
  VARIANT 27: note = F or Y
  VARIANT 32: note = V or absent
  VARIANT 23: note = G, S, T, Y, N, or Q
  VARIANT 5: note = G, S, T, Y, N, or Q
BKXPZBBBXCKXBXCBXBKVXXZBBGBCKCVBR

SEQ ID NO: 21 - protein, 33 aa, synthetic construct
  VARIANT 1: note = K or T
  VARIANT 3: note = absent, M, or Q
  VARIANT 6: note = K or Q
  VARIANT 7: note = R or S
  VARIANT 8: note = R or D
  VARIANT 9: note = R, K, or D
  VARIANT 12: note = T or K
  VARIANT 13: note = Y or F
  VARIANT 14: note = P, A, or R
  VARIANT 16: note = P or R
  VARIANT 17: note = H, K, P, or T
  VARIANT 18: note = P or S
  VARIANT 21: note = R, K, or P
  VARIANT 24: note = I or V
  VARIANT 25: note = K or N
  VARIANT 27: note = F or Y
  VARIANT 32: note = V or absent
  VARIANT 10: note = G, S, T, Y, N, or Q
  VARIANT 28: note = G, S, T, Y, N, or Q
  VARIANT 22: note = D, K or N
BKXPCBBBXZKBBXCBXBKVXXCBBGBZKCVBR

(19) United States
(12) Patent Application Publication
(10) Pub. No.: US 2025/0289855 A1
(43) Pub. Date: Sep. 18, 2025

(54) ANTIMICROBIAL CaNCR PEPTIDE FOLDING VARIANTS

(71) Applicant: Donald Danforth Plant Science Center, ST. LOUIS, MO (US)

(72) Inventors: Dilip M. Shah, Chesterfield, MO (US); Meenakshi Tetorya, St. Louis, MO (US)

(21) Appl. No.: 18/861,420

(22) PCT Filed: May 1, 2023
(86) PCT No.: PCT/US2023/066456

§ 371 Date: Oct. 29, 2024

Related U.S. Application Data
(60) Provisional application No. 63/363,989, filed on May 2, 2022.

(51) Int. Cl. C07K 14/415; A01N 63/50; A01P 1/00; A61K 38/16

(52) U.S. Cl. CPC C07K 14/415; A01N 63/50; A01P 1/00; A61K 38/168

(57) ABSTRACT

Compositions comprising antimicrobial nodule specific cysteine rich (NCR) peptide folding variants (NCR PFV) are disclosed along with compositions. Such compositions comprising the NCR PFV proteins can provide for improved inhibition of fungal and oomycete growth or other desired properties.

### CROSS-REFERENCE TO RELATED APPLICATIONS

The present application claims priority to and the benefit of U.S. Provisional Application No. 63/363,989, filed May 2, 2022, the contents of which are herein incorporated by reference in their entirety.

### REFERENCE TO SEQUENCE LISTING SUBMITTED ELECTRONICALLY

The instant application contains a sequence listing, which has been submitted electronically in XML file format and is hereby incorporated by reference in its entirety. Said XML file created on Apr. 28, 2023, named 225312-528092_Sequence-listing, is 46,989 bytes in size.

### FIELD

The present disclosure relates to compositions comprising antimicrobial nodule specific cysteine rich peptide folding variants (NCR PFV), methods of making such compositions, and methods of use to control microbial infections of plants or animal subjects. The compositions comprising antimicrobial NCR PFV can be applied directly to a plant, human, or animal, applied to a plant in the form of microorganisms that produce the peptides, or the plants can be genetically transformed or edited to produce the peptides or proteins. The present disclosure also relates to recombinant polynucleotides, edited polynucleotides, edited genomes, microorganisms and plants comprising those polynucleotides or genomes, and compositions useful in controlling pathogenic microbes.

### BACKGROUND

Protection of agriculturally important crops from pathogenic microbes (e.g., fungi, including mold, yeast and dimorphic fungi, or oomycetes) is crucial in improving crop yields. Fungal infections are a particular problem in damp climates and can become a major concern during crop storage, where such infections can result in spoilage and contamination of food or feed products with fungal toxins. Unfortunately, modem growing methods, harvesting and storage systems can promote plant pathogen infections.

Certain microbes (e.g., fungi, including yeast, or oomycetes) can also be pathogenic to various vertebrates including humans, fish, and the like. Control of plant pathogens is further complicated by the need to simultaneously control multiple microbes of distinct genera. For example, microbes such as Alternaria; Ascochyta; Botrytis; Cercospora; Colletotrichum; Diplodia; Erysiphe; Fusarium; Gaeumanomyces; Helminthosporium; Macrophomina; Magnaporthe; Nectria; Peronospora; Phoma; Phakopsora, Phymatotrichum; Phytophthora; Plasmopara; Podosphaera; Puccinia; Pythium; Pyrenophora; Pyricularia; Rhizoctonia; Sclerotium; Sclerotinia; Septoria; Thielaviopsis; Uncinula; Venturia; and Verticillium species are all recognized plant pathogens. Consequently, resistant crop plant varieties or antimicrobial agents that control only a limited subset of microbial pathogens can fail to deliver adequate protection under conditions where multiple pathogens are present. It is further anticipated that plant pathogenic microbes can become resistant to existing antimicrobial agents and crop varieties, which can favor the introduction of new microbial control agents with distinct modes of action to combat the resistant microbes.

Certain nodule specific cysteine rich (NCR) peptides and proteins with antimicrobial activity expressed in nodules of Medicago truncatula (Barrel Medic) have been described (WO2010146067). Other NCR peptides from Cicer arietinum (Chickpea) have been described and implicated in the terminal differentiation of endosymbiotic bacteria (Montiel et al., 2016, Molec. Plant Microb. Inter. 29: 210-219). Anti-microbial and anti-oomycete activity of certain NCR peptides has been disclosed in WO2020/146360.

Certain NCR peptides exhibit antimicrobial properties when applied to free-living bacteria and can mediate bacterial cell death and early nodule senescence. Yang, PNAS 114:6848-6853 (2017) and Wang, PNAS 114:6854-6859 (2017). Antimicrobial NCRs (AMPs) are cationic and have conserved cysteine residues that form intramolecular disulfide bonds. Cysteine substitutions or disulfide bond modifications can influence the antimicrobial activity of certain NCR peptides having just 4 conserved cysteine residues. Haag, J. Biol. Chem. 287(14):10791-8 (2012) and Isozumi, Nature Sci. Rep. 11: 9923 (2021).

### SUMMARY

Compositions comprising; (i) an NCR peptide folding variant 1 (NCR PFV1), wherein NCR PFV1 comprises an amino acid sequence having at least 60%, 70%, 80%, 85%, 90%, 92%, 95%, 97%, 98%, 99%, or 100% sequence identity to SEQ ID NO: 1, wherein cysteine residues corresponding to the C4, C10, C15, C23, C28, and C30 cysteine residues of SEQ ID NO: 1 are conserved, wherein said cysteine residues of NCR PFV1 form disulfide bonds between cysteine residues corresponding to C4 to C23, C10 to C28, and C15 to C30, and wherein the composition is substantially free of an NCR peptide folding variant 2 (NCR PFV2) comprising the amino acid sequence of NCR PFV1 and conserved cysteine residues corresponding to the C4, C10, C15, C23, C28, and C30 cysteine residues of SEQ ID NO: 1, wherein said cysteine residues of NCR PFV2 form disulfide bonds between cysteine residues corresponding to C4 to C10, C15 to C30, and C23 to C28; and (ii) an agriculturally, pharmaceutically, or veterinary practicable carrier, diluent, or excipient are provided.

Compositions comprising a nodule-specific cysteine rich NCR peptide folding variant 1 (NCR PFV1), wherein said NCR PFV1 comprises an amino acid sequence having at least 60%, 70%, 80%, 85%, 90%, 92%, 95%, 97%, 98%, 99%, or 100% sequence identity to SEQ ID NO: 1, 2, 3, 4, or 5, wherein cysteine residues C1, C2, C3, C4, C5, and C6 of NCR PFV1 form a first set of disulfide bonds and NCR PFV1 thereby adopts a first conformation that exhibits greater antimicrobial activity as compared to a nodule-specific cysteine rich NCR peptide folding variant 2 (NCR PFV2) that has the same amino acid sequence as NCR PFV1 but forms a distinct second set of disulfide bonds between cysteine residues C1, C2, C3, C4, C5, and C6 and adopts a distinct second conformation, and wherein said composition is substantially free of NCR PFV2, optionally wherein said first set of disulfide bonds comprises a C1-C4 disulfide bond, a C2-C5 disulfide bond, and a C3-C6 disulfide bond are provided.

Compositions comprising a nodule-specific cysteine rich NCR peptide folding variant 1 (NCR PFV1), wherein said NCR PFV1 comprises an amino acid sequence having at least 60%, 70%, 80%, 85%, 90%, 92%, 95%, 97%, 98%, 99%, or 100% sequence identity to SEQ ID NO: 1, 2, 3, 4, or 5, wherein four to six of the cysteine residues corresponding to C1, C2, C3, C4, C5, and C6 of NCR PFV1 are present and form a first set of disulfide bonds, and wherein NCR PFV1 thereby adopts a first conformation that exhibits greater antimicrobial activity, increased protease resistance, or increased protease sensitivity as compared to a nodule-specific cysteine rich NCR peptide folding variant 2 (NCR PFV2) that has the same amino acid sequence as NCR PFV1 but forms a distinct second set of disulfide bonds between four to six of the cysteine residues C1, C2, C3, C4, C5, and C6 and adopts a distinct second conformation, and wherein said composition is substantially free of NCR PFV2, optionally wherein four of the six cysteine residues are present and two of the cysteine residues C1, C2, C3, C4, C5, and C6 are deleted or substituted with a non-cysteine amino acid residue are provided.

Methods of making any of the aforementioned compositions comprising: (i) separating a fraction comprising the NCR peptide folding variant 1 (NCR PFV1) from one or more fractions comprising the NCR peptide folding variant 2 (NCR PFV2) or NCR PFV2 peptide fragments from a mixture comprising NCR PFV1 and NCR PFV2; and (ii) combining the fraction comprising the NCR PFV1 or a further purified preparation therefrom with an agriculturally, pharmaceutically, or veterinary practicable carrier, diluent, or excipient are provided.

Methods for preventing or reducing crop damage and/or post-harvest loss by a plant pathogenic microbe comprising the step of contacting a plant, a plant seed, pre- or post-harvest grain, pre- or post-harvest fruit, pre- or post-harvest vegetable, or other part of said plant with an effective amount of any of the aforementioned compositions are provided.

Methods for treating, preventing, or inhibiting a microbial infection in a subject in need thereof comprising administering to said subject an effective amount of any of the aforementioned compositions are provided.

### BRIEF DESCRIPTION OF THE FIGURES

FIG. 1 shows the HPLC elution profile of NCR13_PFV1 and NCR13_PFV2.

FIG. 2 shows the structures and disulfide bonds of the NCR13_PFV1 and NCR13_PFV2 peptides as determined by NMR.

FIG. 3 shows micrographs of Alternaria alternata and Cercospora sojina under different concentrations of NCR13_PFV1 (Peak 1) and NCR13_PFV2 (Peak 2).

FIG. 4 depicts a drop-inoculation assay showing semi-in planta antifungal activity of NCR13_PFV1 and NCR13_PFV2 in detached N. benthemiana leaves.

FIG. 5 shows semi-in Planta antifungal activity of NCR13_PFV1 against S. sclerotiorum 555 in detached soybean leaves. Representative pictures (under white light and with CropReporter based on the value of potential photosynthetic efficiency Fv/Fm) taken 48 h post-infection showing the antifungal activity of NCR13_PFV1.

FIG. 6 shows semi in planta antifungal activity of NCR13_PFV1 against S. sclerotiorum on soybean pods. Representative pictures taken 72 h post-infection showing the antifungal activity of NCR13_PFV1.

FIG. 7 shows that drop inoculation application of NCR13 PFV1 (0.75 μM) is effective to control B. cinerea growth in tomato fruits at 5 dpi. Bc, Botrytis cinerea.

FIG. 8 shows an alignment of CaNCR13 (SEQ ID NO: 1), CaNCR7 (SEQ ID NO: 3), CaNCR14 (SEQ ID NO: 4), and CaNCR15 (SEQ ID NO: 5) at the conserved C₁, C₂, C₃, C₄, C₅, and C₆ cysteine residues.

### DETAILED DESCRIPTION

### Definitions

The term “and/or” where used herein is to be taken as specific disclosure of each of the two specified features or components with or without the other. Thus, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “include”, “includes”, and “including” are to be construed as at least having the features to which they refer while not excluding any additional unspecified features.

Where a term is provided in the singular, other embodiments described by the plural of that term are also provided.

As used herein, a polynucleotide is said to be “endogenous” to a given cell when it is found in a naturally occurring form and genomic location in the cell.

The phrases “antimicrobial peptide” or “antimicrobial protein” as used herein refer to peptides or proteins which exhibit any one or more of the following characteristics of inhibiting the growth of microbial cells, killing microbial cells, disrupting or retarding stages of the microbial life cycle such as spore germination, sporulation, or mating, and/or disrupting microbial cell infection, penetration or spread within a plant or other susceptible subject, including a human, livestock, poultry, fish, or a companion animal (e.g., dog or cat).

As used herein, the terms “acidic” or “anionic” are used interchangeably to refer to amino acids such as aspartic acid and glutamic acid.

As used herein, the terms “basic” and “cationic” are used interchangeably to refer to amino acids such as arginine, histidine, and lysine.

As used herein, the phrase “consensus sequence” refers to an amino acid, DNA or RNA sequence created by aligning two or more homologous sequences and deriving a new sequence having either the conserved or set of alternative amino acid, deoxyribonucleic acid, or ribonucleic acid residues of the homologous sequences at each position in the created sequence.

The phrases “combating microbial damage”, “combating or controlling microbial damage” or “controlling microbial damage” as used herein refer to reducing damage to a crop plant or crop plant product due to infection by a microbial pathogen. More generally, these phrases refer to reduction in the adverse effects caused by the presence of a pathogenic microbe in the crop plant. Adverse effects of fungal growth are understood to include any type of plant tissue damage or necrosis, any type of plant yield reduction, any reduction in the value of the crop plant product, and/or production of undesirable microbial metabolites or microbial growth by-products including but not limited to mycotoxins.

The term “heterologous”, as used herein in the context of a second polynucleotide that is operably linked to a first polynucleotide, refers to: (i) a second polynucleotide that is derived from a source distinct from the source of the first polynucleotide; (ii) a second polynucleotide derived the same source as the first polynucleotide, where the first, second, or both polynucleotide sequence(s) is/are modified from its/their original form; (iii) a second polynucleotide arranged in an order and/or orientation or in a genomic position or environment with respect to the first polynucleotide that is different than the order and/or orientation in or genomic position or environment of the first and second polynucleotides in a naturally occurring cell; or (iv) the second polynucleotide does not occur in a naturally occurring cell that contains the first polynucleotide. Heterologous polynucleotides include polynucleotides that promote transcription (e.g., promoters and enhancer elements), transcript abundance (e.g., introns, 5′UTR, and 3′UTR), translation, or a combination thereof as well as polynucleotides encoding NCR peptides or localization peptides. In certain embodiments, a nuclear or plastid genome can comprise the first polynucleotide, where the second polynucleotide is heterologous to the nuclear or plastid genome. A “heterologous” polynucleotide that promotes transcription, transcript abundance, translation, or a combination thereof as well as polynucleotides encoding NCR peptides or localization peptides can be autologous to the cell but, however, arranged in an order and/or orientation or in a genomic position or environment that is different than the order and/or orientation in or genomic position or environment in a naturally occurring cell. A polynucleotide that promotes transcription, transcript abundance, translation, or a combination thereof as well as polynucleotides encoding NCR peptides or localization peptides can be heterologous to another polynucleotide when the polynucleotides are not operably linked to one another in a naturally occurring cell. Heterologous peptides or proteins include peptides or proteins that are not found in a cell or organism as the cell or organism occurs in nature. As such, heterologous peptides or proteins include peptides or proteins that are localized in a subcellular location, extracellular location, or expressed in a tissue that is distinct from the subcellular location, extracellular location, or tissue where the peptide or protein is found in a cell or organism as it occurs in nature. Heterologous polynucleotides include polynucleotides that are not found in a cell or organism as the cell or organism occurs in nature.

The term “homolog” as used herein refers to a gene related to a second gene by identity of either the DNA sequences or the encoded protein sequences. Genes that are homologs can be genes separated by the event of speciation (see “ortholog”). Genes that are homologs can also be genes separated by the event of genetic duplication (see “paralog”). Homologs can be from the same or a different organism and in certain embodiments can perform the same biological function in either the same or a different organism.

The phrases “inhibiting growth of a plant pathogenic microbe”, “inhibit microbial growth”, and the like as used herein refers to methods that result in any measurable decrease in microbial growth, where microbial growth includes but is not limited to any measurable decrease in the numbers and/or extent of microbial cells, spores, conidia, or mycelia. As used herein, “inhibiting growth of a plant pathogenic microbe” is also understood to include any measurable decrease in the adverse effects cause by microbial growth in a plant. Adverse effects of microbial growth in a plant include any type of plant tissue damage or necrosis, any type of plant yield reduction, any reduction in the value of the crop plant product, and/or production of undesirable microbial metabolites or microbial growth by-products including but not limited to mycotoxins. As used herein, the phrase “inhibition of microbial growth” and the like, unless otherwise specified, can include inhibition in a plant, human or animal.

As used herein, the terms “microbe”, “microbes”, and “microbial” are used to refer to fungi (including yeast) and oomycetes.

As used herein, the phrase “NCR peptide” or “NCR protein” are used interchangeably refers to any peptide or protein with antimicrobial activity related by any amino acid sequence conservation to a peptide or protein comprising the amino acid sequence of SEQ ID NO: 1, 2, 3, 4, or 5; to peptides or proteins comprising a variant of the amino acid sequence of SEQ ID NO: 1, 2, 3, 4, or 5; to homologs of peptides or proteins comprising the amino acid sequence of SEQ ID NO: 1, 2, 3, 4, or 5; to a fragment of a peptide comprising the amino acid sequence of SEQ ID NO: 1, 2, 3, 4, or 5, a variant thereof, or a homolog thereof; or to any NCR peptide, protein, or fragment thereof set forth in the claims, embodiments, figures, or other disclosure provided herein.

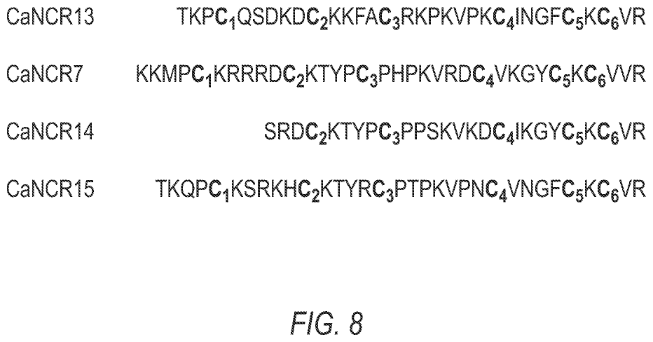

As used herein, the phrase “NCR peptide folding variant” (NCR PFV) refers to an NCR peptide characterized by having a particular set of disulfide bonds between the conserved cysteine residues present in the NCR peptide. Non-limiting examples of an NCR PFV include an NCR peptide comprising SEQ ID NOs: 1, 2, 3, or 4 comprising conserved cysteine residues C₁, C₂, C₃, C₄, C₅, and C₆ in SEQ ID NOs: 1, 2, 3, and 4 (as depicted in FIG. 8) where the NCR PFV comprises disulfide bonds between cysteine residues corresponding to C₁ to C₄, C₂ to C₅, and C₃ to C₆. Non-limiting examples of an NCR PFV include an NCR peptide comprising SEQ ID NO: 1, 2, 3, 4, or 5 comprising 4 to 6 of the conserved cysteine residues C₁, C₂, C₃, C₄, C₅, and C₆ in SEQ ID NOs: 1, 2, 3, and 4 (as depicted in FIG. 8) where an NCR PFV comprises a particular set of disulfide bonds between the cysteine residues present in the NCR peptide. Particular sets of disulfide bonds that can be present in an NCR PFV comprising the 6 conserved cysteines include: (i) C₁ to C₄, C₂ to C₅, and C₃ to C₆ disulfide bonds; and (ii) C₁ to C₂, C₃ to C₆, and C₄ to C₅.

The phrase “operably linked” as used herein refers to the joining of nucleic acid or amino acid sequences such that one sequence can provide a function to a linked sequence. In the context of a promoter, “operably linked” means that the promoter is connected to a sequence of interest such that the transcription of that sequence of interest is controlled and regulated by that promoter. When the sequence of interest encodes a protein that is to be expressed, “operably linked” means that the promoter is linked to the sequence in such a way that the resulting transcript will be efficiently translated. If the linkage of the promoter to the coding sequence is a transcriptional fusion that is to be expressed, the linkage is made so that the first translational initiation codon in the resulting transcript is the initiation codon of the coding sequence. Alternatively, if the linkage of the promoter to the coding sequence is a translational fusion and the encoded protein is to be expressed, the linkage is made so that the first translational initiation codon contained in the 5′untranslated sequence associated with the promoter and the coding sequence is linked such that the resulting translation product is in frame with the translational open reading frame that encodes the protein. Nucleic acid sequences that can be operably linked include sequences that provide gene expression functions (e.g., gene expression elements such as promoters, 5′ untranslated regions, introns, protein coding regions, 3′ untranslated regions, polyadenylation sites, and/or transcriptional terminators), sequences that provide DNA transfer and/or integration functions (e.g., T-DNA border sequences, site specific recombinase recognition sites, integrase recognition sites), sequences that provide for selective functions (e.g., antibiotic resistance markers, biosynthetic genes), sequences that provide scoreable marker functions (e.g., reporter genes), sequences that facilitate in vitro or in vivo manipulations of the sequences (e.g., polylinker sequences, site specific recombination sequences) and sequences that provide replication functions (e.g., bacterial origins of replication, autonomous replication sequences, centromeric sequences). In the context of an amino acid sequence encoding a localization, spacer, linker, or other peptide, “operably linked” means that the peptide is connected to the polyprotein sequence(s) of interest such that it provides a function. Functions of a localization peptide include localization of a protein or peptide of interest (e.g., an NCR peptide) to an extracellular space or subcellular compartment.

The phrases “percent identity” or “sequence identity” as used herein refer to the number of elements (i.e., amino acids or nucleotides) in a sequence that are identical within a defined length of two DNA, RNA or protein segments in an alignment resulting in the maximal number of identical elements, and is calculated by dividing the number of identical elements by the total number of elements in the defined length of the aligned segments and multiplying by 100.

The terms “susceptible microbe (or microbes)”, “susceptible microbial infection”, and the like refer to microbes that infect plants, or human or animal patients or subjects, or microbial infections thereof, that are subjection to inhibition of microbial growth by the NCR PFV disclosed herein.

The phrase “transgenic” refers to an organism or progeny thereof wherein the organism's or progeny organism's DNA of the nuclear or organellar genome contains an inserted exogenous DNA molecule of 10 or more nucleotides in length. Such introduced exogenous DNA molecules can be naturally occurring, non-naturally occurring (e.g., synthetic and/or chimeric), from a heterologous source, or from an autologous source.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Further Description

Compositions comprising desired antimicrobial nodule specific cysteine rich (NCR) peptide folding variants (NCR PFV), methods of making the compositions, and methods of using the compositions are provided herein. In certain embodiments, the NCR PFV can be separated from mixtures comprising the desired NCR PFV and one or more undesired NCR PFV(s) to provide compositions that are enriched for the desired NCR PFV. In certain embodiments, the compositions comprising the desired NCR PFV can exhibit improved unit activity (e.g., units of anti-microbial activity per micrograms or micromoles of NCR protein) relative to compositions comprising a mixture of the desired NCR PFV and one or more reduced activity NCR PFV (raNCR PFV) or the raNCR PFV alone. In certain embodiments, compositions comprising an NCR PFV which exhibits increased protease resistance (e.g., to a serine protease including trypsin) in comparison to the undesired NCR PFV are provided. In other embodiments, compositions comprising a desired NCR PFV can comprise an NCR PFV with increased protease sensitivity (an ipsNCR PFV), and can be separated from an undesired NCR PFV with decreased protease sensitivity (dpsNCR) in comparison to the ipsNCR PFV. Compositions provided herein comprising the desired NCR PFV can be substantially free of the undesired NCR PFV. In certain embodiments, the composition comprising the desired NCR PFV which is substantially free of the undesired NCR PFV can comprise less than about 20%, 10%, 5%, 2%, 1%, or 0.5% of the combined total of the desired NCR PFV and undesired NCR PFV by weight in the composition as the undesired NCR PFV. In certain embodiments, the composition comprising the desired NCR PFV which is substantially free of the undesired NCR PFV can comprise at least about 80%, 90%, 95%, 98%, 99%, or 99.5% of the total NCR peptide in the composition by weight (e.g., total NCR peptide in the form of the desired NCR PFV and one or more undesired NCR PFV) is the desired NCR PFV. In certain embodiments, less than 20%, 10%, 5%, 2%, 1%, or 0.5% of the total NCR peptide in the composition by weight is NCR PFV2 and respectively at least about 80%, 90%, 95%, 98%, 99%, or 99.5% of the total NCR peptide in the composition by weight is NCR PFV1. In certain embodiments, the composition comprises less than about 20%, 10%, 5%, 2%, 1%, or 0.5% by weight of NCR_PFV2 and respectively at least about 80%, 90%, 95%, 98%, 99%, or 99.5% by weight of NCR_PFV1.

Without seeking to be limited by theory, compositions comprising or enriched for NCR PFV with increased protease resistance can be useful in certain methods where an extended shelf life or half-life of the NCR PFV is desirable. However, compositions comprising or enriched for NCR PFV with increased protease sensitivity can be useful in certain methods where a decreased half-life of the NCR PFV is desirable.

NCR peptides which can provide NCR PFV include the following NCR peptides set forth in Table 1 comprising: (i) the amino acid sequence of SEQ ID NO:1, 2, 3, 4, or 5; (ii) a variant of the amino acid sequence of SEQ ID NO:1, 2, 3, 4, or 5 wherein one or more of the hydrophobic, basic, and/or acidic amino acid residues are substituted with hydrophobic, basic, and/or acidic amino acid residues, respectively; or (iii) an amino acid sequence having at least 60%, 70%, 80%, 85%, 90%, 92%, 95%, 97%, 98%, 99%, or 100% sequence identity across the entire length of SEQ ID NO: 1, 2, 3, 4, or 5. NCR peptides include NCR peptides, NCR proteins, and NCR peptide and protein variants set forth in US Patent Application Publication US 20220061333, which is incorporated herein by reference in its entirety.

TABLE 1
NCR peptides

| | | | | % |
|---|---|---|---|---|
| | | | | Hydro- |
| | | | | phobic |
| | Amino acid sequence | | Net | amino |
| Peptide | (SEQ ID NO) | Length | charge | acids |
| NCR13 | TKPCQSDKDCKKFACRKPKVPKC | 32 | +8 | 19 |
| | INGFCKCVR (SEQ ID NO: 1) | | | |
| NCR13- | ATKPCQSDKDCKKFACRKPKVPK | 33 | +8 | 21 |
| Ala | CINGFCKCVR (SEQ ID NO: | | | |
| | 2) | | | |
| NCR7 | KKMPCKRRRDCKTYPCPHPKVR | 34 | +10 | 21 |
| | DCVKGYCKCVVR (SEQ ID NO: | | | |
| | 3) | | | |
| NCR15 | TKQPCKSRKHCKTYRCPTPKVPN | 33 | +9 | 15 |
| | CVNGFCKCVR (SEQ ID NO: | | | |
| | 4) | | | |
| NCR14 | SRDCKTYPCPPSKVKDCIKGYCK | 26 | +5 | 19 |
| | CVR (SEQ ID NO: 5) | | | |

Conserved cysteine residues in NCR peptides can be identified as C₁, C₂, C₃, C₄, C₅, and C₆ in SEQ ID NOs: 1, 2, 3, 4, and 5, where the conserved cysteine residue closest to the amino terminus is C₁ and the cysteine residue closest to the carboxy terminus is C₆ as depicted in FIG. 8. In certain embodiments, the NCR PFV comprises an NCR peptide comprising the peptide sequence of SEQ ID NO: 1, 2, 3, 4, or a variant thereof, wherein the peptide comprises disulfide bonds between cysteine residues corresponding to C₁ to C₄, C₂ to C₅, and C₃ to C₆ of SEQ ID NO: 1, 2, 3, 4, or a variant thereof. In certain embodiments, corresponding raNCR PFV can comprise the peptide sequence of SEQ ID NO: 1, 2, 3, 4, or a variant thereof having disulfide bonds between cysteine residues corresponding to C₁ to C₂, C₃ to C₆, and C₄ to C₅.

In certain embodiments, the NCR PFV comprises an CaNCR13 peptide comprising the peptide sequence of SEQ ID NO: 1, SEQ ID NO: 2, or a variant thereof, wherein the peptide comprises disulfide bonds between cysteine residues corresponding to C₄ to C₂₃ (i.e., C₁ to C₄), C₁₀ to C₂₈ (i.e., C₂ to C₅), and C₁₅ to C₃₀ (i.e., C₃ to C₆) of SEQ ID NO: 1. A corresponding raNCR13 PFV can comprise the peptide sequence of SEQ ID NO: 1 or variant thereof having disulfide bonds between cysteine residues corresponding to C₄ to C₁₀ (i.e., C₁ to C₂), C₁₅ to C₃₀ (i.e., C₃ to C₆), and C₂₃ to C₂₈ (i.e., C₄ to C₅). In certain embodiments, the NCR PFV comprises an CaNCR13-Ala peptide comprising the peptide sequence of SEQ ID NO: 2 or a variant thereof, wherein the peptide comprises disulfide bonds between cysteine residues corresponding to C₁ to C₄, C₂ to C₅, and C₃ to C₆ of SEQ ID NO: 2 or a variant thereof. A corresponding raNCR13 PFV can comprise the peptide sequence of SEQ ID NO: 2 or variant thereof having disulfide bonds between cysteine residues corresponding to C₁ to C₂, C₃ to C₆, and C₄ to C₅.

NCR peptides forming desired NCR PFV provided and used in various embodiments disclosed herein can comprise one or more of the following structural features.

In certain embodiments, a first structural feature of the NCR peptides forming an NCR PFV is a net positive charge at neutral pH. In certain embodiments, the NCR peptides will have a net positive charge at neutral pH of at least +5, +6, +7, +8, +9, or +10. In certain embodiments, the NCR peptides will have a net positive charge at neutral pH of at least +4, +5, +6, +7, +8, +9, or +10 to +12, +13, +14, or +15. In certain embodiments, such net positive charges in NCR peptides can be achieved by methods that include: (i) maintaining cationic (basic) amino acid residues found in NCR peptides including SEQ ID NO: 1, 2, 3, 4, or 5 or substituting such residues with another cationic amino acid residue; (ii) substituting anionic or polar amino acid residues found in NCR peptides including SEQ ID NO: 1, 2, 3, 4, or 5 with a basic amino acid residue; or a combination of (i) and (ii). Examples of such substitutions of cationic amino acid residues in certain NCR peptides include those set forth in Tables 2A, 2B, and 2C. In certain embodiments, such net positive charges in NCR peptides can be achieved by preferentially selecting or substituting a cationic amino acid residue at variable positions in the NCR peptide that correspond to a variable position of SEQ ID NO: 7. For example, a cationic amino acid can be preferentially selected or substituted for any of the variable amino acids at positions X₁ to X₁₈ of SEQ ID NO: 7. In certain embodiments, such selections or substitutions of cationic amino acid residues can be as exemplified in Tables 2A, 2B, and 2C. Substitutions of cationic amino acid residues set forth in Tables 2A, 2B, and 2C for the NCR peptides of SEQ ID NOs: 1, 3, and 4 can also be made in the corresponding amino acid residues of other NCR peptides (e.g., NCR peptides comprising SEQ ID NO: 2 or peptides encompassed by the SEQ ID NO: 7 consensus).

In certain embodiments, a second structural feature of the NCR peptides is a significant percentage of hydrophobic amino acid residues. In certain embodiments, the NCR peptides will comprise at least about 25%, 26%, 28% 30%, 32%, 34%, 36%, 37%, or 38% hydrophobic amino acid residues. In certain embodiments, the NCR peptides will comprise at least about 25%, 26%, 28% 30%, 32%, 34%, or 36% to 37%, 38%, 40%, 42%, or 45% hydrophobic amino acid residues. In certain embodiments, such percentages of hydrophobic amino acids in NCR peptides can be achieved by methods that include: (i) maintaining hydrophobic amino acid residues found in NCR peptides including SEQ ID NO: 1, 2, 3, 4, or 5 or substituting such residues with another hydrophobic amino acid residue or neutral polar amino acid residue; (ii) substituting polar amino acid residues found in NCR peptides including SEQ ID NO: 1, 2, 3, 4, or 5 with a hydrophobic amino acid residue; (iii) substituting neutral polar amino acids for hydrophobic amino acids; or a combination of (i), (ii), and (iii). Examples of such substitutions of hydrophobic amino acid residues in certain NCR peptides include those set forth in Tables 2A, 2B, and 2C. In certain embodiments, such percentages of hydrophobic amino acids in NCR peptides can be achieved by preferentially selecting or substituting a hydrophobic amino acid residue at variable positions in the NCR peptide that correspond to a variable position of SEQ ID NO:7. For example, a hydrophobic amino acid can be preferentially selected or substituted for any of the variable amino acids at positions X₁ to X₁₈ of SEQ ID NO: 7. In certain embodiments, such selections or substitutions of hydrophobic amino acid residues to maintain or increase percentages of hydrophobic amino acids can be as exemplified in Tables 2A, 2B, and 2C. Substitutions set forth in Tables 2A, 2B, and 2C of the NCR peptides of SEQ ID NOs: 1, 3, and 4 can also be made in the corresponding amino acid residues of other NCR peptides (e.g. SEQ ID NO: 2 or NCR peptides containing 4, 5, or 6 of the conserved C₁, C₂, C₃, C₄, C₅, and C₆ cysteine residues set forth in SEQ ID NO: 7 and also shown in FIG. 8). Substitutions of cationic amino acid residues set forth in Tables 2A, 2B, and 2C for the NCR peptides of SEQ ID NO: 1, 3, 4 can also be made in the corresponding amino acid residues of other NCR peptides (e.g., NCR peptides comprising SEQ ID NO: 2, peptides encompassed by the SEQ ID NO: 7 consensus, or NCR peptides containing 4, 5, or 6 of the conserved C₁, C₂, C₃, C₄, C₅, and C₆ cysteine residues set forth in SEQ ID NO: 7 and also shown in FIG. 8).

In certain embodiments, a third structural feature of NCR peptides is the presence of 4, 5, or 6 of the conserved C₁, C₂, C₃, C₄, C₅, and C₆ cysteine residues set forth in SEQ ID NO: 7 and also shown in FIG. 8. In certain embodiments, the NCR PFV will comprise disulfide bonds corresponding to C₁ to C₄, C₂ to C₅, and C₃ to C₆ disulfide bonds in an NCR peptide (e.g., SEQ ID NOs: 1, 2, 3, or 4, or a variant thereof) where the cysteine residue closest to the amino terminus is C₁ and the cysteine residue closest to the carboxy terminus is C₆ as shown in FIG. 8. In any of the aforementioned or other embodiments, the NCR peptide can also comprise 4 of the conserved C₁, C₂, C₃, C₄, C₅, and C₆ cysteine residues. In certain embodiments, one or two of the cysteinyl residues in the NCR peptide can be substituted with a distinct amino acid residue or deleted to provide an NCR peptide with only 4 of the 6 conserved C₁, C₂, C₃, C₄, C₅, and C₆ cysteine residues. In certain embodiments, one or two of the C₁, C₂, C₃, C₄, C₅, or C₆ cysteine residues in the NCR peptide can be substituted with another amino acid residue including a glycine, serine, threonine, tyrosine, asparagine, or glutamine residue to provide an NCR peptide with only 4 of the 6 conserved C₁, C₂, C₃, C₄, C₅, and C₆ cysteine residues. In certain embodiments, one or two of the conserved cysteine residues can be substituted with a serine residue to provide an NCR peptide with only 4 of the 6 conserved C₁, C₂, C₃, C₄, C₅, and C₆ cysteine residues. In certain embodiments, conserved cysteine residues corresponding to a C₁ and a C₄ cysteine residue (e.g., Cys4 and Cys23 in SEQ ID NO: 1) are substituted with another amino acid residue (e.g., serine) to provide an NCR peptide with only 4 of the 6 conserved C₁, C₂, C₃, C₄, C₅, and C₆ cysteine residues. In certain embodiments, conserved cysteine residues corresponding to a C₂ and a C₅ cysteine residue (e.g., Cys10 and Cys28 in SEQ ID NO: 1) are substituted with another amino acid residue (e.g., serine) to provide an NCR peptide with only 4 of the 6 conserved C₁, C₂, C₃, C₄, C₅, and C₆ cysteine residues. Examples of aforementioned substitutions of conserved cysteine residues corresponding to a C₁ and a C₄ cysteine residue or a C₂ and a C₅ cysteine residue include those set forth in SEQ ID NOs: 18 and 19, respectively. Examples of aforementioned substitutions of conserved cysteine residues corresponding to a C₁ and a C₄ cysteine residue or a C₂ and a C₅ cysteine residue also include those set forth in SEQ ID NOs: 20 and 21, respectively. In certain embodiments, an NCR peptide with only 4 of the 6 conserved cysteine residues can comprise a deletion or a substitution of the C₁ and C₆ cysteine residues and will thus comprise the C₂, C₃, C₄, and C₅ cysteine residues. In certain embodiments, an NCR peptide with only 4 of the 6 conserved cysteine residues can comprise a deletion or a substitution of the C₂ or C₆ cysteine residues in an NCR peptide comprising only the C₂, C₃, C₄, C₅, and C₆ cysteine residues (e.g., CaNCR14 (SEQ ID NO: 5) and variants thereof) and thus comprise the C₃, C₄, C₅, and C₆ cysteine residues or the C₂, C₃, C₄, and C₅ cysteine residues.

In certain embodiments, a fourth structural feature in certain NCR peptides is the presence of a core consensus sequence GX₁CKCVX₂R (SEQ ID NO: 11) or a variant thereof comprising an insertion, deletion, and/or substitution of one or more amino acid residues. In certain embodiments, X₁ is F or Y and X₂ is absent or V (e.g. SEQ ID NOs: 8, 9, and 10). In certain embodiments, X₁ is F, Y, W, I, V, A, or M and/or X₂ is absent or F, Y, W, I, V, A, or M in a substitution variant of SEQ ID NO: 11. In certain embodiments, an NCR peptide can also comprise of variant of the SEQ ID NO: 11 core consensus wherein one or both of the cationic residues (K and/or R) is substituted with another cationic amino acid residues (K, R, or H). In certain embodiments, an NCR peptide can also comprise of variant of the SEQ ID NO: 11 core consensus wherein one or both of the conserved tyrosine or valine amino acid residues are substituted with a hydrophobic amino acid (F, W, I, A, or M) or with a tyrosine residue (Y). Examples of substitutions of in the sequence corresponding to the core consensus of the NCR peptides of SEQ ID NOs: 1, 3, and 4 that are set forth in Tables 2A, 2B, and 2C can also be made in the core consensus of other NCR peptides provided herein.

In any of the aforementioned embodiments, the NCR peptide(s) can comprise an amino acid sequence that is at least 60%, 70%, 75%, 80%, 85%, 90%, 95%, 98%, 99%, or 100% identical to SEQ ID NOs: 1, 2, 3, 4, or 5 wherein one or more of the hydrophobic, basic, and/or acidic amino acid residues of SEQ ID NO: 1, 2, 3, 4, or 5 are substituted with other hydrophobic, basic, and/or acidic amino acid residues, respectively. In any of the aforementioned embodiments, the NCR peptide(s) can also comprise an amino acid sequence that is at least 70%, 75%, 80%, 85%, 90%, 95%, 98%, 99%, or 100% identical to SEQ ID NOs: 1, 2, 3, 4, or 5, wherein one or more of the hydrophobic, basic, and/or acidic amino acid residues of SEQ ID NOs: 1, 2, 3, 4, or 5 are substituted with other hydrophobic, basic, and/or acidic amino acid residues, respectively.

In certain embodiments, one or more amino acids in any of the aforementioned or other variant NCR peptide sequences are substituted with another amino acid(s), the charge and polarity of which is similar to that of the original amino acid, i.e., a conservative amino acid substitution. Substitutes for an amino acid within the NCR peptide sequence can be selected from other members of the class to which the originally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (anionic; negatively charged) amino acids such as aspartic acid and glutamic acid; (2) basic (cationic; positively charged) amino acids such as arginine, histidine, and lysine; (3) neutral polar amino acids such as glycine, serine, threonine, cysteine, cystine, tyrosine, asparagine, and glutamine; (4) neutral nonpolar (hydrophobic) amino acids such as alanine, leucine, isoleucine, valine, proline, phenylalanine, tryptophan, and methionine. Conservative amino acid changes within NCR peptide sequences can be made by substituting one amino acid within one of these groups with another amino acid within the same group. Biologically functional equivalents of NCR peptides can have 10 or fewer conservative amino acid changes, seven or fewer conservative amino acid changes, or five, four, three, two, or one conservative amino acid changes. The encoding nucleotide sequence (e.g., gene, plasmid DNA, cDNA, or synthetic DNA) will thus have corresponding base substitutions, permitting it to encode biologically functional equivalent forms of the NCR peptides. Certain semi-conservative substitutions in NCR peptides including: (i) the substitution of a neutral polar amino acid residue with a neutral nonpolar (hydrophobic) amino acid residue; or (ii) the substitution of a neutral nonpolar (hydrophobic) amino acid residue with a neutral polar amino acid residue are also provided. In particular, semi-conservative substitutions of a neutral polar tyrosine residue with a hydrophobic amino acid residue are provided. Semi-conservative substitutions of a hydrophobic amino acid residue with tyrosine residue are also provided. Biologically functional equivalents of NCR peptides can have 10 or fewer semi-conservative amino acid changes, seven or fewer semi-conservative amino acid changes, or five, four, three, two, or one semi-conservative amino acid changes. Non-limiting examples of such conservative substitutions in certain NCR peptides are illustrated in Tables 2A, 2B, and 2C.

Functional fragments of any of the aforementioned NCR PFV can comprise NCR PFV having amino terminal deletions, carboxy terminal deletions, internal deletions, or any combination thereof which leave intact the six conserved cysteine residues of the NCR PFV or contain at least 4 or 5 of the six conserved C₁, C₂, C₃, C₄, C₅, and C₆ cysteine residues. In certain embodiments, the functional fragment can contain at least one, two, three, four, five, six, or seven or more amino acid residue deletions from the amino terminus, the carboxy terminus, an internal region, or any combination thereof. In certain embodiments, antimicrobial fragments of the NCR peptide can comprise at least about 10, 14, 15, 18, or 20 to about 22, 24, 25, 26, 27, or 28 amino acid residues of the C-terminus of the NCR peptide. In any of the aforementioned embodiments, the functional NCR peptide fragment can comprise a peptide encompassed by the consensus sequence of SEQ ID NO: 7. In any of the aforementioned embodiments, the functional NCR peptide fragment can comprise: (i) a conserved core amino acid sequence motif set forth in SEQ ID NOs: 8, 9, 10, or 11; (ii) the amino acid sequence of SEQ ID NOs: 1, 2, 3, 4, or 5; (iii) a variant of a variant of the amino acid sequence of SEQ ID NOs:, wherein 1, 2, 3, 4, or 5 wherein: (a) one or more of the hydrophobic, basic, and/or acidic amino acid residues are substituted with hydrophobic, basic, and/or acidic amino acid residues, respectively; and/or (b) one or more neutral polar amino (e.g., tyrosine) acid residues is substituted with a hydrophobic amino acid residue. Such substitutions of hydrophobic, basic, and/or acidic amino acid residues of the amino acid sequence of SEQ ID NOs: 1 and 2 include those set forth in the corresponding sequences of SEQ ID NO: 1 or those set forth in Tables 2A, 2B, and 2C.

TABLE 2A
NCR13 (SEQ ID NO: 1)
Cationic Amino Acids

| | | | | | | | | | | | | | | | |
|---|---|---|---|---|---|---|---|---|---|---|---|---|---|---|---|
| T | K | P | C | Q | S | D | K | D | C | K | K | F | A | C | R |
| | R | | | | | | R | | | R | R | | | | K |
| | H | | | | | | H | | | H | H | | | | H |

Anionic Amino Acids

| | | | | | | | | | | | | | | | |
|---|---|---|---|---|---|---|---|---|---|---|---|---|---|---|---|
| T | K | P | C | Q | S | D | K | D | C | K | K | F | A | C | R |
| | | | | | | E | | E | | | | | | | |

Hydrophobic Amino Acids

| | | | | | | | | | | | | | | | |
|---|---|---|---|---|---|---|---|---|---|---|---|---|---|---|---|
| T | K | P | C | Q | S | D | K | D | C | K | K | F | A | C | R |
| | | | | | | | | | | | | W | W | | |
| | | | | | | | | | | | | I | I | | |
| | | | | | | | | | | | | M | M | | |
| | | | | | | | | | | | | V | V | | |
| | | | | | | | | | | | | L | L | | |
| | | | | | | | | | | | | A | F | | |

| Cationic Amino Acids | | | | | | | | | | | | | | | |
|---|---|---|---|---|---|---|---|---|---|---|---|---|---|---|---|
| K | P | K | V | P | K | C | I | N | G | F | C | K | C | V | R |
| R | | R | | | R | | | | | | | R | | | K |
| H | | H | | | H | | | | | | | H | | | H |
| Anionic Amino Acids | | | | | | | | | | | | | | | |
| K | P | K | V | P | K | C | I | N | G | F | C | K | C | V | R |
| Hydrophobic Amino Acids | | | | | | | | | | | | | | | |
| K | P | K | V | P | K | C | I | N | G | F | C | K | C | V | R |
| | | | W | | | | W | | | W | | | | W | |
| | | | I | | | | V | | | I | | | | I | |
| | | | M | | | | M | | | M | | | | M | |
| | | | F | | | | F | | | V | | | | F | |
| | | | L | | | | L | | | L | | | | L | |
| | | | A | | | | A | | | A | | | | A | |

TABLE 2B
NCR7 (SEQ ID NO: 3)
Cationic Amino Acids

| | | | | | | | | | | | | | | | | |
|---|---|---|---|---|---|---|---|---|---|---|---|---|---|---|---|---|
| K | K | M | P | C | K | R | R | R | D | C | K | T | Y | P | C | P |
| R | | | | | R | K | K | K | | | R | | | | | |
| H | | | | | H | H | H | H | | | H | | | | | |

Anionic Amino Acids

| | | | | | | | | | | | | | | | | |
|---|---|---|---|---|---|---|---|---|---|---|---|---|---|---|---|---|
| K | K | M | P | C | K | R | R | R | D | C | K | T | Y | P | C | P |
| | | | | | | | | | E | | | | | | | |

Hydrophobic Amino Acids

| | | | | | | | | | | | | | | | | |
|---|---|---|---|---|---|---|---|---|---|---|---|---|---|---|---|---|
| K | K | M | P | C | K | R | R | R | D | C | K | T | Y | P | C | P |
| | | W | | | | | | | | | | | | | | |
| | | F | | | | | | | | | | | | | | |
| | | I | | | | | | | | | | | | | | |
| | | V | | | | | | | | | | | | | | |
| | | L | | | | | | | | | | | | | | |
| | | A | | | | | | | | | | | | | | |

| Cationic Amino Acids | | | | | | | | | | | | | | | | |
|---|---|---|---|---|---|---|---|---|---|---|---|---|---|---|---|---|
| H | P | K | V | R | D | C | V | K | G | Y | C | K | C | V | V | R |
| | | R | | K | | | | R | | | | R | | | | R |
| | | H | | H | | | | H | | | | H | | | | H |
| Anionic Amino Acids | | | | | | | | | | | | | | | | |
| H | P | K | V | R | D | C | V | K | G | Y | C | K | C | V | V | R |
| | | | | | E | | | | | | | | | | | |
| Hydrophobic Amino Acids | | | | | | | | | | | | | | | | |
| H | P | K | V | R | D | C | V | K | G | Y | C | K | C | V | V | R |
| | | | W | | | | W | | | | | | | W | W | |
| | | | F | | | | F | | | | | | | F | F | |
| | | | I | | | | I | | | | | | | I | I | |
| | | | M | | | | M | | | | | | | M | M | |
| | | | L | | | | L | | | | | | | L | L | |
| | | | A | | | | A | | | | | | | A | A | |

TABLE 2C
NCR15 (SEQ ID NO: 4)
Cationic Amino Acids

| | | | | | | | | | | | | | | | | |
|---|---|---|---|---|---|---|---|---|---|---|---|---|---|---|---|---|
| T | K | Q | P | C | K | S | R | K | H | C | K | T | Y | R | C | P |
| | R | | | | R | | K | R | R | | R | | | K | | |
| | H | | | | H | | H | H | K | | H | | | H | | |

Hydrophobic Amino Acids

| | | | | | | | | | | | | | | | | |
|---|---|---|---|---|---|---|---|---|---|---|---|---|---|---|---|---|
| T | K | Q | P | C | K | S | R | K | H | C | K | T | Y | R | C | P |

| Cationic Amino Acids | | | | | | | | | | | | | | | |
|---|---|---|---|---|---|---|---|---|---|---|---|---|---|---|---|
| T | P | K | V | P | N | C | V | N | G | F | C | K | C | V | R |
| | | R | | | | | | | | | | R | | | K |
| | | H | | | | | | | | | | H | | | H |
| Hydrophobic Amino Acids | | | | | | | | | | | | | | | |
| T | P | K | V | P | N | C | V | N | G | F | C | K | C | V | R |
| | | | W | | | | W | | | W | | | | W | |
| | | | F | | | | F | | | V | | | | F | |
| | | | I | | | | I | | | I | | | | I | |
| | | | M | | | | M | | | M | | | | M | |
| | | | L | | | | L | | | L | | | | L | |
| | | | A | | | | A | | | A | | | | A | |

Chimeric NCR peptides comprising portions of any of the aforementioned or other NCR peptides, NCR peptide variants, or NCR fragments can also be used either alone or in the NCR proteins provided herein. In one embodiment, the chimeric NCR peptide can comprise an NCR peptide of SEQ ID NO: 2, 3, 4, or 5 wherein the corresponding core sequence of SEQ ID NO: 5, 8, or 10 found within those NCR peptides is substituted with a distinct core sequence of another NCR peptide. In another embodiment, a C-terminal fragment of an NCR peptide, including a C-terminus of SEQ ID NO: 13, 14, 15, or 16 is substituted with a distinct C-terminal fragment of another NCR peptide.

In certain embodiments, the permeability of a microbial plasma membrane treated with the NCR PFV is increased in comparison to permeability of a microbial plasma membrane treated with a raNCR PFV. Membrane permeability can be measured by a variety of techniques that include dye uptake. Convenient dye uptake assays that can be used to assess changes in in membrane permeability include assays for uptake of Hoechst 33342 (H0342), rhodamine 123, SYTOX™ Green, and the like. These dyes enter into microbial cells only if their plasma membrane has been permeabilized by an NCR peptide, defensin, or other membrane-permeabilizing agent. Without seeking to be limited by theory, in certain embodiments it is believed that the NCR protein comprising an NCR peptide and another antifungal peptide joined by a spacer peptide can provide improved microbial inhibition by increasing the permeability of treated microbial membranes in comparison to microbial membranes treated with an NCR peptide.

In certain embodiments, the NCR PFV can exhibit binding to a phospholipid. In certain embodiments, a desired NCR PFV provided herein can exhibit lower IC50 values against one or more microbial pathogens, improved binding to phospholipids, or any combination thereof in comparison to either: (i) mixtures comprising the desired NCR PFV and reduced activity NCR PFV (raNCR PFV) or (ii) the raNCR PFV. In certain embodiments, the desired NCR PFV can be identified and/or selected for lower IC50 values against one or more microbial pathogens by selecting for NCR PFV that provide for improved phospholipid binding in comparison to in comparison to either: (i) mixtures comprising the NCR PFV and reduced activity NCR PFV (raNCR PFV) or (ii) the raNCR PFV. In certain embodiments, NCR PFV comprise NCR peptides that comprise an amino acid sequence at least 50%, 60%, 70%, 75%, 80%, 85%, 90%, 95%, 98%, 99%, or 100% identical to an amino acid sequence selected from the group consisting of SEQ ID NO: 1, 2, 3, 4, or 5; a variant of the amino acid sequence of SEQ ID NO: 1, 2, 3, 4, or 5, wherein one or more of the hydrophobic, basic, and/or acidic amino acid residues are substituted with hydrophobic, basic, and/or acidic amino acid residues, respectively; a variant of the amino acid sequence of SEQ ID NO:1, 2, 3, 4, or 5, wherein one or more neutral polar amino (e.g., tyrosine) acid residues is substituted with a hydrophobic amino acid residue; and/or wherein one or more hydrophobic amino acid residues is substituted with a neutral polar amino acid residue (e.g., tyrosine); functional fragments thereof, and chimeras thereof. Suitable assays for determining improved phospholipid include protein-lipid overlay assays (e.g., Dowler et al., 2002, Sci STKE. 2002 Apr. 23; 2002(129):16), surface plasmon resonance assays (e.g., Baron and Pauron, 2014, Bio-protocol 4(18): e1237), biotin capture lipid affinity assays (e.g., Davidson et al., 2006, Lipid Research, 47, 440-449), titration calorimetry assays (e.g., Miller and Cistola, 1993 Molecular and Cellular Biochemistry, 123(1): 29-37), and the like.

In other embodiments, the NCR peptide or protein encoding nucleotide sequence can be synthesized de novo from an NCR peptide sequence disclosed herein. The sequence of the peptide or protein-encoding nucleotide sequence can be deduced from the NCR peptide sequence (e.g., SEQ ID NOs: 1-5, or a variant thereof) through use of the genetic code. Computer programs such as “BackTranslate” (GCG™ Package, Acclerys, Inc. San Diego, CA) can be used to convert a peptide sequence to the corresponding nucleotide sequence that encodes the peptide.

Infection of certain plants with certain plant pathogen microbes can result in distinctive effects on plant growth that are readily observed. Consequently, one can distinguish plants treated with an NCR PFV from a plant treated with a control lacking an NCR peptide or comprising the corresponding raNCR PFV by simply challenging such plants with pathogenic plant microbes and observing reduction of the symptoms normally associated with such infections. Such observations are facilitated by co-infecting otherwise identical, control plants treated with a control lacking an NCR peptide or comprising the corresponding raNCR PFV. Identification of treated plants that control or combat microbial infection can be based on observation of decreased disease symptoms, measurement of the decreased microbial growth in the infected plant (e.g., by determining the numbers of colony forming units per gram of infected tissue) and/or by measurement of the amount of mycotoxin present in infected plant tissue. The use of microbial disease severity assays and colony formation assays in conjunction with expression assays to identify transgenic MsDefl-expressing potato plants that are resistant to Verticillium dahliae has been described (U.S. Pat. No. 6,916,970 and Gao et al., 2000, Nature Biotechnology 18, 1307-1310). It is similarly anticipated that a variety of NCR PFV treated plants that combat or control microbial pathogens can be identified by scoring treated plants for resistance to microbial pathogens that infect those plants. Examples of NCR PFV-conferred microbial resistance that can be assayed by observing reductions in disease symptoms or reductions in microbial growth include resistance of treated corn to Fusarium verticillioides, Fusarium moniliforme, Colletotrichum graminicola, Stenocarpella maydis, and/or Cercospora zeae-maydis; resistance of treated wheat to head blight (Fusarium graminearum), powdery mildew (Erysiphe graminis f. sp. tritici), stripe rust, stem rust or leaf rust (Puccinia tritici); resistance of treated cotton to Fusarium oxysporum and Verticillium dahlia; resistance of treated rice to Magnaporthe oryzae and Rhizoctonia solani, and resistance of treated soybean to Asian Soybean rust (Phakopsora pachyrhizi), Phytophthora Root Rot (Phytophthora sp.), White Mold (Sclerotinia sp.), Sudden Death Syndrome (Fusarium virguliforme) and/or Brown Stem Rot (Phialophora gregata).

NCR PFV treated plants that have plant pathogenic microbe inhibitory amounts of an NCR PFV can also be identified by measuring decreases in the adverse effects cause by microbial growth in such plants. Such decreases can be ascertained by comparing the extent of the adverse effect in an NCR PFV treated plant relative to an otherwise identical, control plant treated with a control composition lacking the NCR PFV or comprising the corresponding raNCR PFV. Adverse effects of microbial growth in a plant that can be measured include any type of plant tissue damage or necrosis, any type of plant yield reduction, any reduction in the value of the crop plant product, and/or production of undesirable microbial metabolites or microbial growth by-products including, but not limited to, mycotoxins. Mycotoxins comprise a number of toxic molecules produced by microbial species, including but not limited to polyketides (including aflatoxins, demethylsterigmatocystin, O-methylsterigmatocystin, etc.), fumonisins, alperisins (e.g., Als A2, Bls B2), sphingofungins (A, B, C and D), trichothecenes, fumifungins, and the like. Methods of quantitating mycotoxin levels are widely documented. Moreover, commercial kits for measurement of the mycotoxins such as aflatoxin, fumonisin, deoxynivalenol, and zearalenone are also available (VICAM, Watertown, MA, USA).

Plants of interest include both food crop plants and biofuels or energy crop plants. The plants, parts thereof, and harvested parts thereof (e.g., grain, fruits, and vegetables) to which the methods and compositions disclosed herein can be applied can refer to plants, parts thereof, and harvested parts thereof that are either directly edible, or which produce edible products (e.g., customarily used to feed humans either directly, or indirectly through animals). Plants, parts thereof, and harvested parts thereof used herein include cereal crop (e.g., wheat, rice, maize (corn), barley, oats, sorghum, rye, and millet), legumes (e.g., peanuts, chickpeas, lentils, kidney beans, soybeans, lima beans); root or tuber crop (e.g., potatoes, sweet potatoes, and cassavas); oil crops (e.g., canola (rapeseed), wheat, peanuts, palm, coconuts, safflower, cottonseed, sunflower, flax, and olive); sugar crops (e.g., sugar cane and sugar beets); fruit crops (e.g., bananas, oranges, apples, pears, breadfruit, pineapples, strawberries, grapes, and cherries); vegetable crops and tubers (e.g., tomatoes, lettuce, carrots, melons, asparagus, Brassica sp.); nut crops (e.g., cashews, peanuts, walnuts, pistachio nuts, almonds); forage and turf grasses; forage legumes (e.g., alfalfa, clover); drug crops (e.g., Cannabis sp., coffee, cocoa, kola nut, poppy); spice and flavoring crops (e.g., vanilla, sage, thyme, anise, saffron, menthol, peppermint, spearmint, coriander), fiber crop (e.g., cotton or hemp), and biofuel crops (e.g., castor bean, Camelina, switchgrass, Miscanthus, and Jatropha) plants, parts thereof, and harvested parts thereof.

Expression of NCR peptides in yeast and filamentous fungi to produce NCR PFV is specifically contemplated herein. The construction of expression vectors for production of heterologous proteins in various yeast genera is well established. In general, such expression vectors typically comprise a promoter that is operably linked to a sequence of interest that is operably linked to a polyadenylation or terminator region. Examples of yeast genera that have been used to express heterologous genes include Candida, Kluveromyces, Hansuela, Pichia, Saccharomyces, Schizosaccharomyces, and Yarrowia. A general description of expression vectors and transformation systems for Saccharomyces is found in Kingsman et al (1985) Biotechnol Genet Eng Rev. 3:377-416. Expression vectors and transformation systems useful for yeasts other than Saccharomyces are described in Reiser et al (1990) Adv Biochem Eng Biotechnol.; 43:75-102. Other examples of fungal systems which can be adapted for use in expressing NCR peptides include filamentous fungal systems such as Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes and Trichoderma systems (e.g. U.S. Pat. Nos. 11,046,736 and 11,180,767, both incorporated herein by reference in their entireties). Other systems which can be adapted for use in expressing NCR peptides include Chrysosporium lucknowense systems (e.g., U.S. Pat. Nos. 8,871,493, and 9,175,296, incorporated herein by reference in its entirety).

Expression of NCR peptides in bacterial cells including Escherichia sp. (e.g., E. coli) to produce NCR PFV is also specifically contemplated herein. Systems for expressing proteins which comprise disulfide bonds can be adapted for expression of the NCR peptides in E. coli include those disclosed in US Patent Application US20200172915, which is incorporated herein by reference in its entirety, and in Berkmen, M. Protein Expr Purif. 2012; 82(1):240-51. doi: 10.1016/j.pep.2011.10.009. Other systems useful for expression of proteins which comprise disulfide bonds can be adapted for expression of the NCR peptides in E. coli include those disclosed in Kuddus et al., (2017) Biotechnol Prog 233:1520-1528. doi: 10.1002/btpr.Protein Science 2508; Kiedzierska et al. (2008) Protein Expr Purif 60, 82-88; Chang et al., (2015) Amino Acids 47, 579-587; Buchko et al. (2018) (2018) Protein Science 27, 1611-1623; and Marques et al., (2008) J Appl Microbiol 106, 1640-1648; Pazgier, M., Lubkowski, J. (2006) Protein Expr Pur 49, 1-8.

In general, the promoter and polyadenylation region are selected based on their operability in a given bacterial, yeast, or fungal host. For example, the AOX1 or AOX2 promoters of Pichia can be used in conjunction with the AOX1, AOX2, p40, or p76 polyadenylation sequences of Pichia to express a heterologous protein such as an NCR peptide. Both the AOX1 and AOX2 promoters are particularly useful in Pichia as both promoters provide for abundant expression of the linked heterologous gene when induced by addition of methanol to the growth medium. The use of these Pichia promoters and polyadenylation sequences is described in U.S. Pat. No. 4,855,231, which is expressly incorporated herein by reference in its entirety. Similarly, the Hansuela MOX, DHAS, or FMDH promoters can be used to express heterologous proteins such as NCR in Hansuela. The MOX, DHAS, or FMDH promoters are particularly useful in Hansuela as these promoters provide for abundant expression of the linked heterologous gene when induced by addition of methanol to the growth medium. The use of the MOX and DHAS promoters in Hansuela is described in U.S. Pat. No. 5,741,672, while the use of the FMDH promoter in Hansuela is described in U.S. Pat. No. 5,389,525, each of which is expressly incorporated herein by reference in its entirety. For Kluveromyces, a Lactase promoter and polyadenylation sequence can be used to express heterologous genes such as NCR. Expression of heterologous genes that are operably linked to the Lactase promoter and polyadenylation sequence is achieved by growing Kluveromyces in the presence of galactose. The use of the Lactase promoter and polyadenylation sequences in Kluveromyces is described in U.S. Pat. No. 6,602,682, which is expressly incorporated herein by reference in its entirety.

Yeast, bacterial, or fungal expression vectors that provide for secretion of heterologous proteins such as NCR into the growth medium by transformed yeast or fungi are also contemplated. Secretion of the mature NCR peptide is typically achieved by operable linkage of a signal peptide sequence or a signal peptide and propeptide sequence to the mature NCR protein- or peptide-encoding sequence. Examples of useful signal peptides for secretion of heterologous proteins in yeast include but are not limited to an alpha-factor signal peptide, an invertase signal peptide, and a PHO1 signal peptide, all of which are derived from yeast. The alpha-factor signal peptide is typically derived from Saccharomyces, Kluveromyces, or Candida, while the PHO1 signal peptide is derived from Pichia.

A particularly useful signal peptide sequence or signal peptide and propeptide sequence for secretion of proteins in yeast is derived from the S. cerevisiae alpha-factor, and is described in U.S. Pat. Nos. 4,546,082, 4,588,684, 4,870,008, and 5,602,034, each of which is expressly incorporated herein by reference in its entirety. The S. cerevisiae alpha-factor signal peptide and propeptide sequence consist of amino acids 1-83 of the primary, unprocessed translation product of the S. cerevisiae alpha mating factor gene (GenBank Accession Number: P01149). In certain embodiments, the signal peptide sequence of the alpha-mating factor comprising amino acids 1 to about 19 to 23 of the alpha-mating factor proprotein can be directly linked to the N-terminus of the mature NCR protein to provide for secretion of mature NCR protein. In this case, the signal peptide is cleaved from the mature NCR protein in the course of the secretion process. Alternatively, the signal peptide and propeptide of the alpha mating factor can be operably linked to the mature NCR encoding sequence via a cleavage site sequence. This cleavage site sequence can comprise a variety of sequences that provide for proteolytic processing of the leader sequence and gene of interest. In the native S. cerevisiae alpha mating factor gene the s cleavage site sequence corresponds to amino acid residues 84-89 and is represented by the sequence Lys84-Arg85-Glu86-Ala87-Glu88-Ala 89 (SEQ ID NO: 17). The sequence Lys-Arg corresponds to a KEX2 protease recognition site while the Glu-Ala-Glu-Ala sequence corresponds to a duplicated dipeptidylaminopeptidase or STE13 recognition site. In certain embodiments, a DNA fragment encoding the 89 amino acid S. cerevisiae alpha factor signal, propeptide coding region, and entire native spacer coding region (i.e., the N-terminal 89 amino acid residues of the alpha mating factor precursor protein containing both the Lys-Arg KEX2 protease cleavage site at residues 84 and 85 as well as the Glu-Ala-Glu-Ala dipeptidylaminopeptidase or STE13 recognition site at residues 86-89) is operably linked to the sequence encoding the mature NCR protein. When the N-terminal 89 amino acids of the alpha mating factor precursor protein are fused to the N-terminus of a heterologous protein such as NCR, the propeptide sequence is typically dissociated from the heterologous protein via the cleavage by endogenous yeast proteases at either the KEX2 or STE13 recognition sites. In other embodiments, a DNA fragment encoding the smaller 85 amino acid Saccharomyces cerevisiae alpha factor signal peptide, propeptide, and KEX2 spacer element (i.e., the N-terminal 85 amino acid residues of the alpha mating factor precursor protein containing just the Lys-Arg KEX2 protease cleavage site at residues 84 and 85) is operably linked to the sequence encoding the mature NCR protein. When the N-terminal 85 amino acids of the alpha mating factor precursor protein are fused to the N-terminus of a heterologous protein such as NCR, the propeptide sequence is typically dissociated from the heterologous protein via cleavage by endogenous yeast proteases at the KEX2 recognition site. The NCR protein can thus be expressed without the glu-ala repeats.

To obtain transformed yeast that express NCR peptides, the yeast NCR expression cassettes (e.g., yeast promoter, yeast signal peptide encoding sequence, mature NCR protein sequence, and polyadenylation sequence) are typically combined with other sequences that provide for selection of transformed yeast. Examples of useful selectable marker genes include genes encoding a ADE protein, a HIS5 protein, a HIS4 protein, a LEU2 protein, a URA3 protein, ARG4 protein, a TRP1 protein, a LYS2 protein, a protein conferring resistance to a bleomycin or phleomycin antibiotic, a protein conferring resistance to chloramphenicol, a protein conferring resistance to G418 or geneticin, a protein conferring resistance to hygromycin, a protein conferring resistance to methotrexate, an a AR04-OFP protein, and a FZF1-4 protein. Similar electable marker cassettes that confer resistance to antibiotics or rescue auxotrophic traits can be used in bacterial or fungal systems.

DNA molecules comprising the yeast NCR expression cassettes and selectable marker genes are introduced into yeast cells by techniques such as transfection into yeast spheroplasts or electroporation. In certain embodiments, the DNA molecules comprising the yeast NCR expression cassettes and selectable marker genes are introduced as linear DNA fragments that are integrated into the genome of the transformed yeast host cell. Integration can occur at random sites in the yeast host cell genome or at specific sites in the yeast host cell genome. Integration at specific sites in the yeast host cell genome is typically accomplished by homologous recombination between sequences contained in the expression vector and sequences in the yeast host cell genome. Homologous recombination is typically accomplished by linearizing the expression vector within the homologous sequence (for example, within the AOX1 promoter sequence of a Pichia expression vector when integrating the expression vector into the endogenous AOX1 gene in the Pichia host cell). In other embodiments, the yeast expression cassettes can also comprise additional sequences such as autonomous replication sequences (ARS) that provide for the replication of DNA containing the expression cassette as an extrachromosomal (non-integrated) element. Such extra-chromosomal elements are typically maintained in yeast cells by continuous selection for the presence of the linked selectable marker gene. Yeast artificial chromosomes (YACs) containing sequences that provide for replication and mitotic transmission are another type of vector that can be used to maintain the DNA construct in a yeast host.

Yeast, bacterial, or fungal cells transformed with the yeast or fungal NCR expression cassettes can be used to produce mixtures of NCR PFV that can be used to obtain preparation enriched for a desired NCR PFV. The desired NCR PFV can be used as antimicrobial agents, to produce antimicrobial compositions that can be applied to plants or foodstuffs, or to produce antimicrobial compositions that can be administered to human or non-human subjects. Methods of producing mixtures of NCR PFV can typically first comprise the step of culturing yeast, bacterial, or fungal cells transformed with NCR expression cassettes under conditions wherein the yeast cells express a mature NCR molecule. In general, the conditions where the yeast bacterial or fungal cells express the mature NCR molecules are conditions that allow for or specifically induce expression of the yeast promoter that is operably linked to the NCR coding sequence in the yeast expression cassette. When the yeast is Pichia and the signal-peptide/MD gene is under the control of an AOX1 or AOX2 promoter, addition of methanol to the growth medium will provide for expression of mature NCR protein. Similarly, when the yeast is Hansuela and the signal-peptide/MD gene is under the control of a MOX, DHAS, or FMDH promoter, addition of methanol to the growth medium will provide for expression of mature NCR protein. Alternatively, when the yeast is Kluveromyces and the signal-peptide/De/5 gene is under the control of a Lactase promoter, addition of galactose to the growth medium will provide for expression of mature NCR protein.

Once the transformed yeast, bacterial, or fungal culture has been incubated under culture conditions that provide for expression of mature NCR peptide for a sufficient period of time, the desired NCR molecule PFV can be separated from any undesired NCR PFV isolated from the culture as a mixture. A sufficient time period can be determined by periodically harvesting portions or aliquots of the culture and assaying for the presence of desired and/or undesired NCR PFV. Analytical assays such as HPLC be used to monitor desired NCR PFV and undesired NCR PFV production. In certain embodiments, culture incubation periods can be optimized for production of the desired NCR PFV such that the culture is enriched for the desired NCR PFV.

Isolation of the desired NCR PFV from the culture can be partial or complete. For NCR expression vectors where a yeast, bacterial, or fungal signal peptide is operably linked to the sequence encoding the mature NCR protein, a mixture comprising the desired NCR PFV and any raNCR PFV or other undesired NCR PFV (e.g., increased or decreased protease resistance) can be recovered from the yeast cell culture medium, bacterial periplasm, or fungal cell culture medium. In certain embodiments, yeast or fungal cell culture medium that contains the mature NCR protein in the form of both a desired NCR PFV and an undesired NCR PFV can be separated from the yeast or fungal cells by centrifugation or filtration, thus providing a composition comprising a mixture of the desired and undesired NCR PFV. In certain embodiments, preparations of bacterial periplasmic proteins that contains the mature NCR protein in the form of both a desired NCR PFV and an undesired NCR PFV can be obtained by methods including physical force, pressure, osmotic shock, or chemical (e.g., EDTA+heat or detergent) treatment, thus providing a composition comprising a mixture of the desired and undesired NCR PFV. Such methods for isolation of bacterial periplasmic proteins can be adapted from methods disclosed for isolation of other periplasmic proteins (e.g., as in Schimek et al., (2020) Biotechnology progress, 36(5), e2999. doi.org/10.1002/btpr.2999). Yeast or fungal cell culture medium or preparations of bacterial periplasmic proteins that contain the mature NCR protein can be further processed by any combination of dialysis and/or concentration techniques (e.g., precipitation, lyophilization, filtration) to produce a composition containing one or more NCR PFV (e.g. a mixture of the desired and undesired NCR PFV). In certain embodiments, filtration size-exclusion membranes can be used to enrich for a fraction containing a mixture of desired and undesired NCR PFV. Combinations of various isolation techniques can also be employed to produce a mixture of NCR PFV. For example, the cell culture medium can be separated from the cells by centrifugation and dialyzed or adjusted. In certain embodiments, a buffer for dialysis or adjustment is a 25 mM sodium acetate buffer at about pH4.5-pH6.0. This dialysate is then subjected to ion-exchange chromatography. For example, a cation-exchange resin such as CM-Sephadex C-25 equilibrated with a 25 mM sodium acetate buffer at about pH6.0 can be used. NCR protein bound to the cation exchange resin is washed and then eluted. For example, the aforementioned column is washed with 25 mM sodium acetate buffer at about pH6.0 and subsequently eluted in 1M NaCl, 50 mM Tris, pH7.6. Fractions containing the NCR protein are identified by an assay or by UV absorbance and then concentrated by a size-cutoff filtration membrane. The concentrated NCR protein is then dialyzed to obtain an essentially or substantially pure NCR protein in a buffer. Buffers include buffers such as 10 mM Tris, pH 7.6.

Production of compositions comprising or enriched for the desired NCR PFV can be obtained by methods that provide for separation of the desired NCR PFV from the undesired NCR PFV(s). In certain embodiments, such methods can comprise standard protein separation techniques, which enrich for the desired NCR PFV based on different chemical properties besides mass (e.g., the hydrodynamic radius of different NCR PFV and/or the charge characteristics of the different NCR PFV). In certain embodiments, chromatographic techniques such as sedimentation, size exclusion chromatography (SEC), ion-exchange chromatography, and/or affinity chromatography can be used to produce a composition enriched for the desired NCR PFV. Methods for separating proteins having different conformations by SEC which have been disclosed can be adapted for use in separating a desired from one or more undesired NCR PFV(s) (La Verde et al., Bio Protoc. 2017 Apr. 20; 7(8):e2230. doi: 10.21769/BioProtoc.2230). In certain embodiments, the methods can provide for a composition comprising a desired NCR PFV where the composition comprises less than 5%, 2%, 1%, 0.5%, 0.25%, or 0.1% of one or more undesirable NCR PFV(s) by weight. In certain embodiments, the methods can provide for a composition comprising a desired NCR PFV where less than 5%, 2%, 1%, or 0.5% of the combined total of the desired NCR PFV and one or more undesired NCR PFV(s) by weight in the composition is/are the undesired NCR PFV(s). In certain embodiments, the methods can provide for a composition comprising a desired NCR PFV where at least about 80%, 90%, 95%, 98%, 99%, or 99.5% of the total NCR peptide by weight or molarity in the composition is the desired NCR PFV. In certain embodiments, the methods can provide a composition where less than 20%, 10%, 5%, 2%, 1%, or 0.5% of the total NCR peptide in the composition by weight is undesired NCR PFV or wherein at least about 80%, 90%, 95%, 98%, 99%, or 99.5% of the total NCR peptide in the composition by weight is the desired NCR PFV. Total NCR peptide by weight in the composition can be determined by quantifying the combined total amount of desired and undesired NCR PFV in the composition. Quantification of total NCR peptides and/or an isolated NCR PFV can be accomplished by a variety of methods including immunoassays (e.g., ELISAs, RIA where a purified NCR peptide is used to establish a reference curve) or mass spectroscopy (Zhang et al. Methods Mol Biol. 2010; 673:211-222. doi:10.1007/978-1-60761-842-3_13).

In certain embodiments, compositions enriched for desired NCR PFV can be obtained by methods, which exploit the relative sensitivity of a desired NCR PFV to a protease (e.g., a serine protease such as trypsin). In embodiments where the desired NCR PFV exhibits increased resistance to the protease (e.g., at a given protease concentration), mixtures comprising the desired and undesired NCR PFV can be treated with the protease at a concentration which will cleave the undesired NCR PFV yet leave the desired NCR PFV intact. The intact NCR PFV can then be separated from the resultant fragments of the NCR peptide produced by cleavage of the undesired NCR PFV by methods including size-exclusion membrane filtration, SEC, and/or IEC. Suitable proteases for such methods can include a trypsin family serine protease. In certain embodiments, the serine protease will cleave an undesired NCR PFV at the carboxy terminus of an arginine or lysine residue in that undesired NCR PFV under non-denaturing conditions. In certain embodiments, the trypsin family serine protease is a recombinant bovine, porcine, human, or microbial trypsin, optionally wherein the microbial trypsin is a Streptomycete trypsin or variant thereof. In certain embodiments, about 6.25, 10, or 12.5 to about 15, 20, or 25 BAEE units/mL of trypsin (e.g., Sigma-Aldrich Catalog No. T1426, St. Louis, MO) can be incubated with the NCR PFV for about 1, 2, or 3 to about 4, 5, or 6 hours at 37° C. in 60 mM monobasic sodium phosphate buffer at pH 7.5 to cleave the undesired NCR PFV yet leave the desired NCR PFV intact. One BAEE (Na-Benzoyl-L-arginine ethyl ester hydrochloride) trypsin substrate unit will produce a delta A253 (change in absorbance at 253 nm) of 0.001 per minute with BAEE as substrate at pH 7.6 at 25 deg C. in a reaction volume=3.2 ml (1 cm light path).

The analysis of different NCR PFV to assign particular disulfide bonds can be performed by mass spectrometry-based methods (Weinfurtner, in Oxidative Folding of Proteins: Basic Principles, Cellular Regulation and Engineering, 2018, pp. 81-98 DOI: 10.1039/9781788013253-00081; Tang and Speicher, Current protocols in protein science vol. 96, 1 (2019): e86. doi:10.1002/cpps.86).

Also provided are antimicrobial compositions for agricultural, pharmaceutical, or veterinary use, comprising either an antimicrobial plant, antimicrobial human or veterinary, pathogenic microbe inhibitory amount (“antimicrobial effective amount”) of one or more the present isolated, purified antimicrobial NCR PFV peptides, or biologically functional equivalents thereof. Such compositions can comprise one, or any combination of, NCR peptides disclosed herein, and an agriculturally, pharmaceutically, or veterinary-practicably acceptable carrier, diluent, or excipient. As indicated below, other components relevant in agricultural and therapeutic contexts can be included in such compositions as well. The antimicrobial compositions can be used for inhibiting the growth of, or killing, NCR protein- or peptide-susceptible pathogenic microbes associated with plant, human or animal microbial infections. Such antimicrobial compositions can be formulated for topical administration, and applied topically to either plants, the plant environment (including soil), or humans or animals.

Agricultural compositions comprising any of the present NCR molecules alone, or in any combination, can be formulated as described in, for example, Winnacker-Kuchler (1986) Chemical Technology, Fourth Edition, Volume 7, Hanser Verlag, Munich; van Falkenberg (1972-1973) Pesticide Formulations, Second Edition, Marcel Dekker, N.Y.; and K. Martens (1979) Spray Drying Handbook, Third Edition, G. Goodwin, Ltd., London. Formulation aids, such as carriers, inert materials, surfactants, solvents, and other additives are also well known in the art, and are described, for example, in Watkins, Handbook of Insecticide Dust Diluents and Carriers, Second Edition, Darland Books, Caldwell, N.J., and Winnacker-Kuchler (1986) Chemical Technology, Fourth Edition, Volume 7, Hanser Verlag, Munich. Using these formulations it is also possible to prepare compositions where a desired NCR PFV is combined with other pesticidally active substances, fertilizers, and/or growth regulators, etc., in a composition (e.g., a finished formulation or tank mixes.

Whether alone or in combination with other active agents, the present antimicrobial NCR PFV peptides can be applied at a concentration in the range of from about 0.1 g/ml to about 100 mg/ml, or from about 5 g/ml to about 5 mg/ml, at a pH in the range of from about 3.0 to about 9.0. Such compositions can be buffered using, for example, phosphate buffers between about 1 mM and 1 M, about 10 mM to about 100 mM, or about 15 mM to about 50 mM. In the case of low buffer concentrations, a salt can be added to increase the ionic strength. In certain embodiments, NaCl in the range of from about 1 mM to about 1 M, or about 10 mM to about 100 mM, can be added.

Numerous conventional microbial antibiotics and chemical fungicides with which the present NCR PFV can be combined are described in Worthington and Walker (1983) The Pesticide Manual, Seventh Edition, British Crop Protection Council. These include, for example, polyoxines, nikkomycines, carboxy amides, aromatic carbohydrates, carboxines, morpholines, inhibitors of sterol biosynthesis, and organophosphorous compounds. In addition, azole, triazole, and/or echinocandin fungicides can also be used. Other active ingredients that can be formulated in combination with the present antimicrobial peptides and proteins include, for example, insecticides, attractants, sterilizing agents, acaricides, nematicides, and herbicides. U.S. Pat. No. 5,421,839, which is incorporated herein by reference in its entirety, contains a comprehensive summary of the many active agents with which substances such as the present antimicrobial NCR peptides and proteins can be formulated.

The compositions provided herein can be applied in an antimicrobial effective amount, which will vary depending on such factors as, for example, the specific fungal pathogen to be controlled, the specific plant (and plant part or soil) to be treated, and the method of applying the compositions that comprise a desired NCR PFV.

Desired NCR PFV and biologically functional equivalents and compositions comprising the same provided herein (e.g., in the following numbered embodiments), can be used to inhibit the growth of a wide variety of susceptible microbes in plants. In certain embodiments, growth of microbes in the following genera or species can be inhibited: Alternaria (e.g., Alternaria brassicicola; Altemaria solani); Ascochyta (e.g., Ascochyta pisi); Aspergillus (e.g., Aspergillus flavus; Aspergillus fumigatus); Botrytis (e.g., Botrytis cinerea); Cercospora (e.g., Cercospora kikuchii; Cercospora zeae-maydis); Colletotrichum (e.g., Colletotrichum lindemuthianum); Diplodia (e.g., Diplodia maydis); Erysiphe (e.g., Erysiphe graminis f. sp. graminis; Erysiphe graminis f. sp. hordei); Fusarium (e.g., Fusarium nivale; Fusarium oxysporum; Fusarium graminearum; Fusarium culmorum; Fusarium solani; Fusarium moniliforme; Fusarium roseum); Gaeumanomyces (e.g., Gaeumanomyces graminis tsp. tritici); Helminthosporium (e.g., Helminthosporium turcicum; Helminthosporium carbonum; Helminthosporium maydis); Macrophomina (e.g., Macrophomina phaseolina; Magnaporthe grisea); Nectria (e.g., Nectria heamatococca); Peronospora (e.g., Peronospora manshurica; Peronospora tabacina); Phakopsora (e.g., Phakopsora pachyrhizi); Phoma (e.g., Phoma betae); Phymatotrichum (e.g., Phymatotrichum omnivorum); Phytophthora (e.g., Phytophthora cinnamomi; Phytophthora cactorum; Phytophthora phaseoli; Phytophthora parasitica; Phytophthora citrophthora; Phytophthora sojae; Phytophthora infestans); Plasmopara (e.g., Plasmopara viticola); Podosphaera (e.g., Podosphaera leucotricha); Puccinia (e.g., Puccinia sorghi; Puccinia striiformis; Puccinia graminis f. sp. tritici; Puccinia asparagi; Puccinia recondita; Puccinia arachidis); Pythium (e.g., Pythium aphanidermatum; Pythium ultimum); Pyrenophora (e.g., Pyrenophora tritici-repentens); Pyricularia (e.g., Pyricularia oryzae); Rhizoctonia (e.g., Rhizoctonia solani; Rhizoctonia cerealis); Sclerotium (e.g., Sclerotium rolfsii); Sclerotinia (e.g., Sclerotinia sclerotiorum); Septoria (e.g., Septoria lycopersici; Septoria glycines; Septoria nodorum; Septoria tritici); Thielaviopsis (e.g., Thielaviopsis basicola); Uncinula (e.g., Uncinula necator); Venturia (e.g., Venturia inaequalis); and Verticillium (e.g., Verticillium dahliae; Verticillium albo-atrum).

Pharmaceutical or veterinary compositions that comprise an antimicrobial effective amount of a desired NCR PFV and a pharmaceutically acceptable or veterinary practicable carrier are also provided. Such pharmaceutical or veterinary compositions can be used for inhibiting the growth of, or killing, susceptible pathogenic microbes that infect humans or animals, i.e., treating such fungal infections by administering to a patient or other subject in need thereof. In certain embodiments, compositions comprising NCR PFV, can be formulated by methods adapted from those described in Remington: The Science and Practice of Pharmacy (2005), 21st Edition, University of the Sciences in Philadelphia, Lippincott Williams & Wilkins. In certain embodiments, the compositions can contain NCR PFV at concentrations in the range of from about 0.1 μg per ml to about 100 mg per ml, or about 5 μg per ml to about 5 mg per ml, at a pH in the range of from about 3.0 to about 9.0. Such compositions can be buffered using, for example, phosphate buffers at a concentration of about 1 mM to about 1 M, about 10 mM to about 100 mM, or about 15 mM to 50 mM. In the case of low buffer concentrations, a salt can be added to increase the ionic strength. In certain embodiments, NaCl in the range of about 1 mM to about 1 M, or about 10 mM to about 100 mM, can be added.

The NCR PFV can be formulated alone or can formulated in combination with other conventional antimicrobial therapeutic compounds such as, by way of non-limiting example, polyene antimicrobials; imidazole, triazole, and thiazole antimicrobials; allylamines; and echinocandins that are routinely used in human and veterinary medicine.

Administration of the compositions that comprise NCR PFV to a human or animal subject in need thereof can be accomplished via a variety of routes that include topical application, enteral, parenteral, and/or intravenous introduction.

### Embodiments

The following numbered embodiments form part of the disclosure.

1a. A composition comprising;
(i) an NCR peptide folding variant 1 (NCR PFV1), wherein NCR PFV1 comprises an amino acid sequence having at least 60%, 70%, 80%, 85%, 90%, 92%, 95%, 97%, 98%, 99%, or 100% sequence identity to SEQ ID NO: 1, wherein cysteine residues corresponding to the C4, C10, C15, C23, C28, and C30 cysteine residues of SEQ ID NO: 1 are conserved, wherein said cysteine residues of NCR PFV1 form disulfide bonds between cysteine residues corresponding to C4 to C23, C10 to C28, and C15 to C30, and wherein the composition is substantially free of an NCR peptide folding variant 2 (NCR PFV2) comprising the amino acid sequence of NCR PFV1 and conserved cysteine residues corresponding to the C4, C10, C15, C23, C28, and C30 cysteine residues of SEQ ID NO: 1, wherein said cysteine residues of NCR PFV2 form disulfide bonds between cysteine residues corresponding to C4 to C10, C15 to C30, and C23 to C28; and
(ii) an agriculturally, pharmaceutically, or veterinary practicable carrier, diluent, or excipient.

1b. A composition comprising a nodule-specific cysteine rich NCR peptide folding variant 1 (NCR PFV1), wherein said NCR PFV1 comprises an amino acid sequence having at least 60%, 70%, 80%, 85%, 90%, 92%, 95%, 97%, 98%, 99%, or 100% sequence identity to SEQ ID NO: 1, 2, 3, 4, or 5, wherein cysteine residues C₁, C₂, C₃, C₄, C₅, and C₆ of NCR PFV1 form a first set of disulfide bonds and NCR PFV1 thereby adopts a first conformation that exhibits greater antimicrobial activity as compared to a nodule-specific cysteine rich NCR peptide folding variant 2 (NCR PFV2) that has the same amino acid sequence as NCR PFV1 but forms a distinct second set of disulfide bonds between cysteine residues C₁, C₂, C₃, C₄, C₅, and C₆ and adopts a distinct second conformation, and wherein said composition is substantially free of NCR PFV2, optionally wherein said first set of disulfide bonds comprises a C₁-C₄ disulfide bond, a C₂-C₅ disulfide bond, and a C₃-C₆ disulfide bond.

1c. A composition comprising a nodule-specific cysteine rich NCR peptide folding variant 1 (NCR PFV1), wherein said NCR PFV1 comprises an amino acid sequence having at least 60%, 70%, 80%, 85%, 90%, 92%, 95%, 97%, 98%, 99%, or 100% sequence identity to SEQ ID NOs: 1, 2, 3, 4, 5, 18, 19, 20, or 21, wherein four to six of the cysteine residues corresponding to C₁, C₂, C₃, C₄, C₅, and C₆ of NCR PFV1 are present and form a first set of disulfide bonds, and wherein NCR PFV1 thereby adopts a first conformation that exhibits greater antimicrobial activity, increased protease resistance, or increased protease sensitivity as compared to a nodule-specific cysteine rich NCR peptide folding variant 2 (NCR PFV2) that has the same amino acid sequence as NCR PFV1 but forms a distinct second set of disulfide bonds between four to six of the cysteine residues C₁, C₂, C₃, C₄, C₅, and C₆ and adopts a distinct second conformation, and wherein said composition is substantially free of NCR PFV2, optionally wherein four of the six cysteine residues are present and two of the cysteine residues C₁, C₂, C₃, C₄, C₅, and C₆ are deleted or substituted with a non-cysteine amino acid residue.

2. The composition of embodiment 1a, 1b, or 1c, wherein:
(i) wherein said NCR PFV1 exhibits greater protease resistance than said NCR PFV2;
(ii) wherein said NCR PFV1 comprises an amino acid sequence having an isoelectric point of pH8.0 to pH12.0; and/or
(ii) wherein said NCR PFV1 said NCR peptide comprises one or more substitutions of a hydrophobic, basic, and/or acidic amino acid residues of SEQ ID NO: 1, 2, 3, 4, or 5 with hydrophobic, basic, and/or acidic amino acid residues, respectively; and/or (ii) an amino acid sequence having at least 80%, 85%, 90%, 92%, 95%, 97%, 98%, or 99% sequence identity across the entire length of SEQ ID NO: 1, 2, 3, 4, or 5.

3. The composition of any one of embodiments 1a, 1b, 1c, or 2, wherein less than 5%, 2%, 1%, or 0.5% of the total NCR peptide in the composition by weight is NCR PFV2 or wherein at least about 80%, 90%, 95%, 98%, 99%, or 99.5% of the total NCR peptide in the composition by weight is NCR PFV1.

4. The composition of embodiment 1a, 1b, 1c, or 2, wherein less than 5%, 2%, 1%, or 0.5% of the combined total of NCR PFV1 and NCR PFV2 by weight is NCR PFV2.

5. A method of making the composition of any one of embodiments 1a, 1b, 1c, 2, 3, or 4 comprising:
(i) separating a fraction comprising the NCR peptide folding variant 1 (NCR PFV1) from one or more fractions comprising the NCR peptide folding variant 2 (NCR PFV2) or NCR PFV2 peptide fragments from a mixture comprising NCR PFV1 and NCR PFV2; and
(ii) combining the fraction comprising the NCR PFV1 or a further purified preparation therefrom with an agriculturally, pharmaceutically, or veterinary practicable carrier, diluent, or excipient, thereby making the composition.

6. The method of embodiment 5, wherein the method further comprises obtaining the mixture by culturing microorganisms comprising a recombinant polynucleotide comprising a promoter which is operably linked to a polynucleotide encoding a signal peptide which is operably linked to a polypeptide comprising an amino acid sequence having at least 60%, 70%, 80%, 85%, 90%, 92%, 95%, 97%, 98%, 99%, or 100% sequence identity to SEQ ID NO: 1, 2, 3, 4, or 5 and separating the microorganisms from a fermentation broth comprising the mixture.

7. The method of embodiment 5, wherein the method further comprises obtaining the mixture by disrupting a culture of microorganisms comprising a recombinant polynucleotide comprising a promoter which is operably linked to a polynucleotide encoding a polypeptide comprising an amino acid sequence having at least 60%, 70%, 80%, 85%, 90%, 92%, 95%, 97%, 98%, 99%, or 100% sequence identity to SEQ ID NO: 1, 2, 3, 4, or 5 to: (a) obtain a cell lysate and separating insoluble debris in the cell lysate from an aqueous fraction comprising the mixture, optionally wherein the microorganisms comprise Candida, Kluveromyces, Hansenula, Pichia, Saccharomyces, Schizosaccharomyces, or Yarrowia, Myceliophthora, or Escherichia microorganisms; or (b) obtain a periplasmic protein preparation comprising the mixture; wherein the microorganism is optionally an Escherichia microorganism.

8. The method of embodiment 5 or 6, wherein the separating further comprises treating the mixture comprising NCR PFV1 and NCR PFV2 with a protease at a concentration sufficient to produce the NCR PFV2 peptide fragments but insufficient to produce NCR PFV1 peptide fragments.

9. The method of embodiment 8, wherein the protease comprises a trypsin family serine protease which cleaves NCR PFV2 at the carboxy terminus of an NCR PFV2 arginine or lysine residue under non-denaturing conditions.

10. The method of embodiment 9, wherein the trypsin family serine protease is a recombinant bovine, porcine, human, or microbial trypsin, optionally wherein the microbial trypsin is a Streptomycete trypsin or variant thereof.

11. The method of embodiment 8, wherein the NCR PFV1 is separated from the NCR PFV2 peptide fragments by size-exclusion filtration or size-exclusion chromatography (SEC).

12. The method of any one of embodiments 5 to 11, wherein the separating is effected by chromatography, optionally wherein the chromatography is SEC or IEC.

13. A method for preventing or reducing crop damage and/or post-harvest loss by a plant pathogenic microbe comprising the step of contacting a plant, a plant seed, pre- or post-harvest grain, pre- or post-harvest fruit, pre- or post-harvest vegetable, or other part of said plant with an effective amount of the composition of any one of embodiments 1 to 4.

14. The method of embodiment 13, wherein the plant pathogenic microbe is a Fusarium sp., Alternaria sp., Verticillium sp., Phytophthora sp., Colletotrichum sp., Botrytis sp., Cercospora sp., Phakopsora sp. Rhizoctonia sp., Sclerotinia sp., Pythium sp., Phoma sp., Leptosphaeria sp., Gaeumannomyces sp., Puccinia sp. Septoria sp., Penicillium sp., Lasiodiplodia sp., Phomopsis sp., Mycosphaerella sp., Golovinomyces sp., Erisyphe sp., Albugo sp., Setosphaeria sp., Cochliobolus sp., Helminthosporium sp., Diplodia sp. or Stenocarpella sp.

15. The method of embodiment 13 or 14, wherein the plant, a plant seed, pre- or post-harvest grain, pre- or post-harvest fruit, or pre- or post-harvest vegetable is a cereal crop, legume crop, root or tuber crop, oil crop, fruit crop, vegetable crop, nut crop, forage or turf grass crop, forage legume, drug crop, spice or flavoring crop, fiber crop, or biofuel crop plant, a plant seed, pre- or post-harvest grain, pre- or post-harvest fruit, or pre- or post-harvest vegetable.

16. The method of embodiment 15, the plant, a plant seed, pre- or post-harvest grain, pre- or post-harvest fruit, or pre- or post-harvest vegetable is a wheat, rice, corn, barley, oat, sorghum, rye, millet, peanut, chickpea, pigeon pea, lentil, kidney bean, soybean, lima bean, potato, sweet potato, cassava, canola, peanut, palm, coconut, safflower, cotton, sunflower, flax, olive, sugarcane, sugarbeet, banana, orange, apple, pear, breadfruit, pineapple, strawberry, grape, cherry, coffee, coco, tomato, lettuce, carrot, melon, asparagus, Brassica sp., cashew, walnut, pistachio, almond, alfalfa, clover, castor, Camelina, pennycress, switchgrass, Miscanthus, or Jatropha plant, a plant seed, pre- or post-harvest grain, pre- or post-harvest fruit, or pre- or post-harvest vegetable.

17. A medical device comprising the device and the composition of any one of embodiments 1 to 4, wherein the device comprises at least one surface that is topically coated and/or impregnated with the composition.

18. The medical device of embodiment 17, wherein said device is a stent, a catheter, a contact lens, a condom, a patch, or a diaphragm.

19. A method for treating, preventing, or inhibiting a microbial infection in a subject in need thereof comprising administering to said subject an effective amount of the composition of any one of embodiments 1 to 4.

20. The method of embodiment 19, wherein said administration comprises topical, enteral, parenteral, and/or intravenous introduction of the composition.

21. The method of embodiment 19 or 20, wherein the subject is a human, livestock, poultry, fish, or a companion animal.

22. The method of any one of embodiments 19 to 21, wherein the microbial infection is of a mucosal membrane, eye, skin, and/or a nail and the composition is applied to the mucosal membrane, eye, skin, and/or nail.

23. The method of any one of embodiments 19 to 22, wherein the microbial infection is by a dermatophyte, and wherein the dermatophyte is optionally selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.

24. The method of any one of embodiments 19 to 22, wherein the microbial infection is by an Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, or Blastomyces species.

25. The method of any one of embodiments 19 to 22, wherein the microbial infection is by a Candida species and wherein the Candida species is Candida albicans (C. albicans), C. auris, C. glabrata, C parasilosis, C. tropicalis, or C. krusei.

26. The composition of any one of embodiments 1 to 4 for use in a method of treating, preventing, or inhibiting microbial infection in a subject in need thereof.

27. The composition of embodiment 26, wherein the subject is a human, livestock, poultry, fish, or a companion animal.

28. A plant part that is at least partly coated with the composition of any one of embodiments 1 to 4.

29. The plant part of embodiment 28, wherein the part is a seed or a pre- or post-harvest grain.

30. The plant part of embodiment 28 or 29, wherein the plant part is a pre- or post-harvest fruit, vegetable, or flower.

31. The plant part of any one of embodiments 28 to 30, wherein the plant part is a cereal crop, legume crop, root or tuber crop, oil crop, fruit crop, vegetable crop, nut crop, forage or turf grass crop, forage legume, drug crop, spice or flavoring crop, fiber crop, or biofuel crop plant part.

32. The plant part of embodiment 31, the plant part is a wheat, rice, corn, barley, oat, sorghum, rye, millet, peanut, chickpea, pigeon pea, lentil, kidney bean, soybean, lima bean, potato, sweet potato, cassava, canola, peanut, palm, coconut, safflower, cotton, sunflower, flax, olive, sugarcane, sugarbeet, banana, orange, apple, pear, breadfruit, pineapple, strawberry, grape, cherry, coffee, coco, tomato, lettuce, carrot, melon, asparagus, Brassica sp., cashew, walnut, pistachio, almond, alfalfa, clover, castor, Camelina, pennycress, switchgrass, Miscanthus, or Jatropha plant part.

### EXAMPLES

### Example 1. Heterologous Expression and Purification of NCR13_PFV1 and NCR13_PFV2

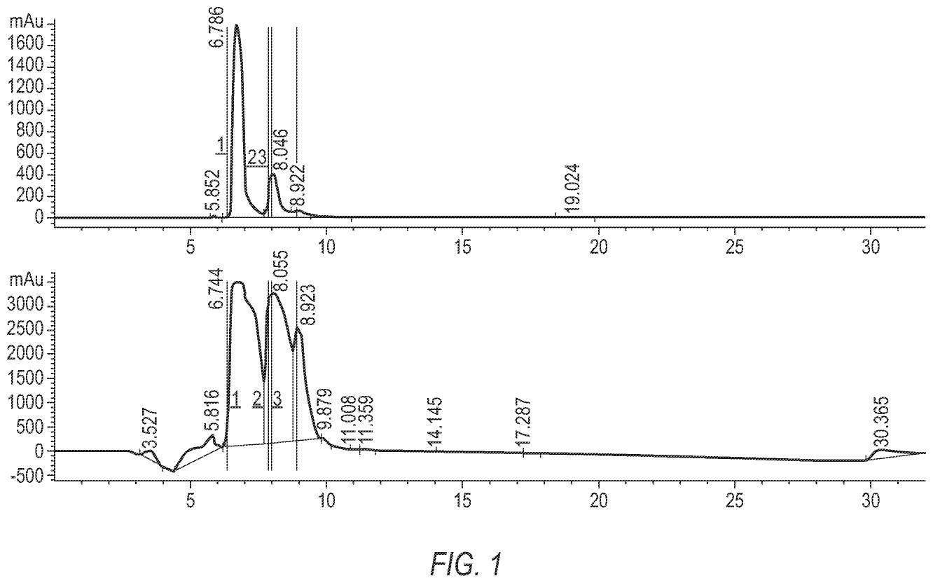

A synthetic NCR13 gene (SEQ ID NO: 6) with an additional codon for Ala at the 5′ end was obtained from Genscript Biotech (Piscataway, NJ, USA). For expression in Pichia pastoris X33, synthetic coding sequence encoding the NCR13-Ala peptide (SEQ ID NO: 2) was cloned into a SacI linearized pPICZα-A integration vector (Invitrogen, Carlsbad, CA) at XhoI and XbaI restriction sites in-frame with the α-mating factor secretion signal sequence containing KEX2 cleavage site without the Glu-Ala repeats. The peptides were purified using the CM-Sephadex C-25 cation-exchange chromatography and C18 reverse-phase HPLC as described previously with minor modifications (Sagaram et al. (2011) PLoS One. doi: 10.1371/journal.pone.0018550). After induction, cells were harvested by centrifugation at 6,000 rpm at 4° C. for 20 minutes, and the pH of the supernatant was adjusted to 6.0. The cation-exchange resin (CM-Sephadex C-25, Sigma, Cat no: C25120) previously equilibrated with binding buffer (25 mM sodium acetate, pH 6.0) was added to the supernatant and incubated overnight at 4° C. at 110 rpm. After collecting and washing the resin with a binding buffer, the bound proteins were eluted with the elution buffer (1M NaCl, 50 mM Tris, pH 7.6) using AKTA FPLC. The FPLC fraction containing peptide was concentrated using an Amicon Ultra-15 Centrifugal filter units. The concentrated fractions were dialyzed against 10 mM Tris, pH 7.6 and further purified by reverse phase C18-HPLC. Two peaks were observed (FIG. 1) and fractions containing each peak were collected using the method provided in the Agilent HPLC system manual. HPLC fractions containing Peak1 and Peak2 peptides were lyophilized and resuspended in nuclease-free water. NCR13-Ala peptides in these two peaks are designated NCR13_PFV1 and NCR13_PFV2. The HPLC profile of NCR13 revealed two peaks. NCR13_PFV1 eluted at ˜6.8 min and PCR13_PFV2 eluted at ˜8.1 min (FIG. 1). The concentration of NCR13_PFV1 and NCR13_PFV2 was determined using the BCA assay performed according to the manufacturer's protocol (Thermo-Fisher Scientific). The purity and size of NCR13_PFV1 and NCR13_PFV2 were verified by sodium dodecyl sulfate-polyacrylamide gel electrophoresis (SDS-PAGE).

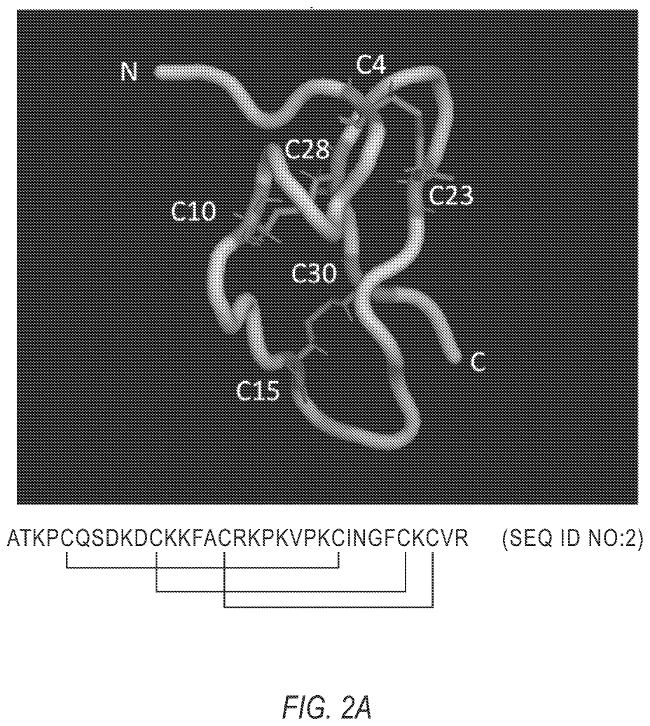

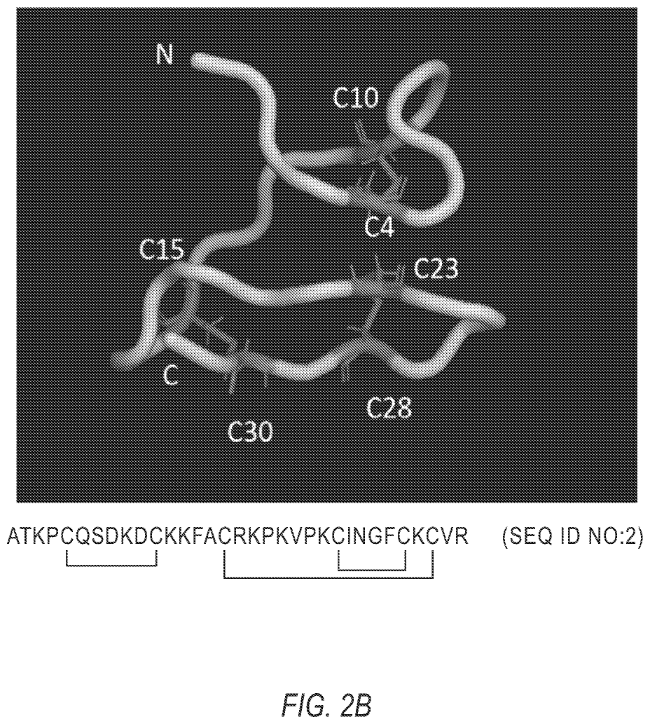

The NCR13_PFV1 and NCR13_PFV2 peptides were subjected to NMR analysis to determine the disulfide bonds present in each peptide. NCR13_PFV1 had disulfide bonds between cysteine residues corresponding to C4 and C23, C10 and C28, and between cysteine residues C15 to C30 in SEQ ID NO: 1 (FIG. 2A). NCR13_PFV2 had disulfide bonds between cysteine residues corresponding to C4 to C10, C15 to C30, and C23 to C28 in SEQ ID NO: 1 (FIG. 2B).

### Example 2. Fungal Cultures and Spore Suspensions

The fungal strain of Botrytis cinerea T-4 was cultured in 20% V8 growth medium. Alternaria alternata was grown on potato dextrose agar. Fungal spores were harvested by flooding the fungal growth plates with sterile water. The spore suspension was filtered through two layers of Miracloth, centrifuged at 13,600 rpm for 1 min, washed, and re-suspended in low-salt Synthetic Fungal Medium (SFM) (U.S. Pat. No. 6,916,970). The spore suspension was adjusted to equivalent spore density using a hemocytometer.

Cercospora sojina and Alternaria alternata conidia were harvested from a fully grown culture plate by gently dislodging the conidia with an L-spreader in the presence of 5 ml sterile water. To remove mycelial fragments, the spore suspension was filtered through three layers of miracloth and washed twice with sterile water at 13,000 rpm for 2 minutes. The spore concentration was adjusted to 1×10⁵ spores/ml using a hemocytometer.

Sclerotinia sclerotiorum (S. sclerotiorum) 555 fungal cultures were grown on potato dextrose agar (PDA) medium (Difco Laboratories Inc., Detroit, MI, USA) at room temperature.

### Example 3. Determination of the Minimum Inhibitory Concentration (MIC) of Ncr13_PFV1 and Ncr13_PFV2 in Absence and Presence of Cations

Antifungal activity of NCR13_PFV1 and NCR13_PFV2 was assessed at different concentrations using a 2-fold dilution series of each peptide. Antifungal activity of each peptide was determined spectrophotometrically using the 96-well plate assay (Sagaram et al., (2011) PLoS One. doi: 10.1371/journal.pone.0018550; Sagaram et al., (2013). PLoS One. 2013 Dec. 4; 8(12):e82485. doi: 10.1371/journal.pone.0082485). Forty-five microliters of each peptide at different concentrations was added to each well of the microtiter plate containing 45 μL of ˜10⁵ spores/ml spore suspension. The quantitative fungal growth inhibition was determined by measuring the absorbance at 595 nm using a microplate reader (Tecan Infinite M200 ProTecan Systems Inc., San Jose, CA) after 48 h. The fungal cell viability was determined by the resazurin cell viability assay (Li et al., 2019, MPMI 32, 1649-1664; Velivelli et al., 2020, doi.org/10.1073/pnas.2003526117). After incubation of the pathogen/peptide mixture for 48 h, 1 of 0.1% resazurin solution was added to each well. After incubation of the mixture overnight, a change in the color of the resazurin dye from blue to pink or colorless indicated the presence of live fungal cells. The MIC for each peptide was the lowest concentration of each peptide at which no change in blue color occurred. Using this protocol, the MIC value of NCR13_PFV1 and NCR13_PFV2 was also determined in presence of 100 mM NaCl and 2 mM CaCl₂).

The MIC of NCR13_PFV1 and NCR13_PFV2 against B. cinerea was determined (Table 3). MIC assays were carried out in 2×SFM. NCR13_PFV1 and NCR13_PFV2 inhibited the growth of this fungus at MIC values of 0.375 or lower and 1.5 μM, respectively, indicating fourfold or greater antifungal activity of NCR13_PFV1. It has been hypothesized antifungal peptides significantly lose their antifungal activity in presence of cations since electrostatic interactions between a positively charged peptide and negatively charged fungal membranes are significantly weakened in presence of cations. We therefore determined antifungal activity of NCR13_PFV1 and NCR13_PFV2 in SFM supplemented with 100 mM NaCl or 2 mM CaCl₂) (Table 3). Both peptides lost their activity in presence of either NaCl or CaCl₂ indicating that the antifungal activity of these peptides is salt-sensitive.

The antifungal assay results revealed that NCR13_PFV1 was superior in inhibiting the growth of C. sojina at concentration as low as 0.187 μM and A. alternata at 0.75 μM, whereas NCR13_PFV2 inhibited these two pathogens at 1.5 μM and 3 μM, respectively (Table 3).

TABLE 3
Antifungal activity of NCR13_PFV1 and
NCR13_PFV2 against fungal pathogens.

| | | SFM | SM + | SFM + |
|---|---|---|---|---|
| | | MIC | 100 mM | 2 mM |
| Pathogen | Peptide | (μM) | NaCl | CaCl₂ |
| Botrytis cinerea | NCR13_PFV1 | 0.375 or | Inactive | Inactive |
| | | lower | | |
| | NCR13_PFV2 | 1.5 | Inactive | Inactive |
| Cercospora | NCR13_PFV1 | 0.187 or | ND | ND |
| sojina | | lower | | |
| | NCR13_PFV2 | 1.5 | ND | ND |
| Alternaria | NCR13_PFV1 | 0.75 | ND | ND |
| alternata | NCR13_PFV2 | 3.0 | ND | ND |
| Sclerotinia | NCR13_PFV1 | 6.0 | ND | ND |
| sclerotiorum | NCR13_PFV2 | 24.0 | ND | ND |

SFM = Synthetic Fungal Medium,
ND = Not determined

### Example 4. Microscopic Observation of the Antifungal Activity of NCR13_PFV1 and Ncr13_PFV2 Against Alternaria alternata and Cercospora sojina

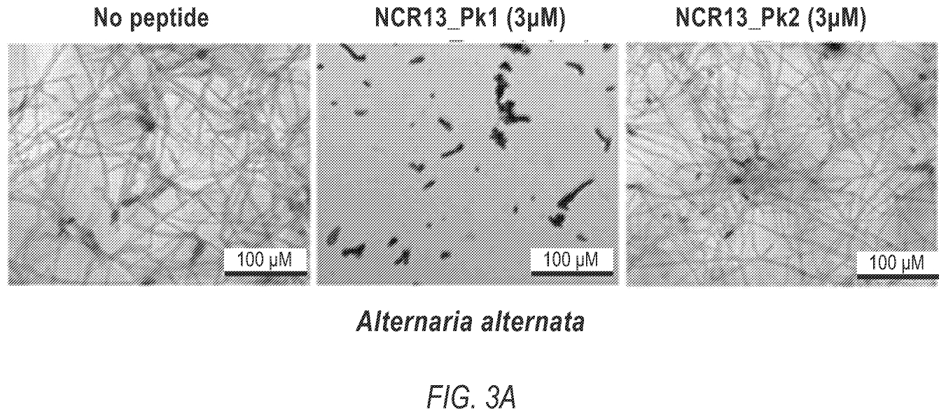

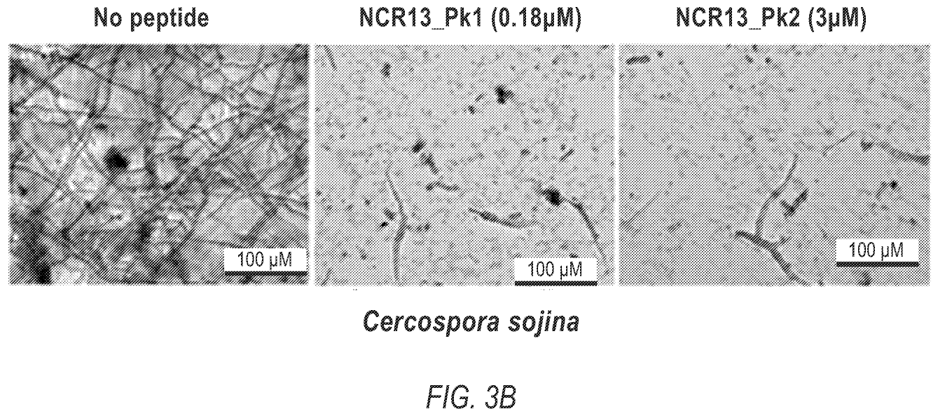

The presence of germinating conidia/mycelial fragments was observed under a light microscope at 20× magnification. In the case of A. alternata, NCR13_PFV1 showed complete inhibition of conidial germination at 3 μM, whereas NCR13_PFV2 showed significant mycelial growth at 3 μM. (FIG. 3A). In contrast, NCR13_PFV1 completely inhibited C. sojina growth at 0.18 μM, whereas NCR13_PFV2 inhibited C. sojina growth only at 3 μM, which is nearly 17 times higher concentration than NCR13_PFV2 (FIG. 3B).

### Example 5. In Vitro Antifungal Activity of NCR13_PFV1 Against Sclerotinia sclerotiorum (S. sclerotiorum) 555

The antifungal activity of NCR13_PFV1 against S. sclerotiorum 555 was measured in an in vitro assay using 24-wells plate assay. Mycelial plugs (1 mm in diameter) cut from the margin of two day-old actively growing colony were transferred from potato dextrose agar media to a series of wells containing 125 microliter (L) of 2×SFM medium (U.S. Pat. No. 6,916,970), with 125 μL of NCR13_PFV1 at varying concentrations of 3, 6, 12 and 24 μM. The plates were incubated at room temperature for 2 days prior to assessment. The mycelium diameter growth was measured 2 dpi using ImageJ software. NCR13 PFV1 peptide effects were calculated as a percentage from the difference between growth of the control and treated mycelium using the formula:

IG (%)=(Dc−Dt)/Dc×100; where IG is inhibition of growth, Dc is the mycelium diameter (mm) of the control and Dt is the mycelium diameter (mm) of the peptide-treated dishes.

After 2 days post infection (dpi), media treated with no peptide showed large S. sclerotiorum 555 growth. However, S. sclerotiorum 555 growth was progressively reduced in size with increasing concentrations of NCR13_PFV1. NCR13_PFV1 exhibited potent fungicidal activity against S. sclerotiorum 5S5 in vitro the minimum inhibitory concentration (MIC) of 6 μM.

### Example 6. NCR13_PFV1 Performed Better than NCR13_PFV2 Against the Gray Mold Disease in Semi-In Planta Assays

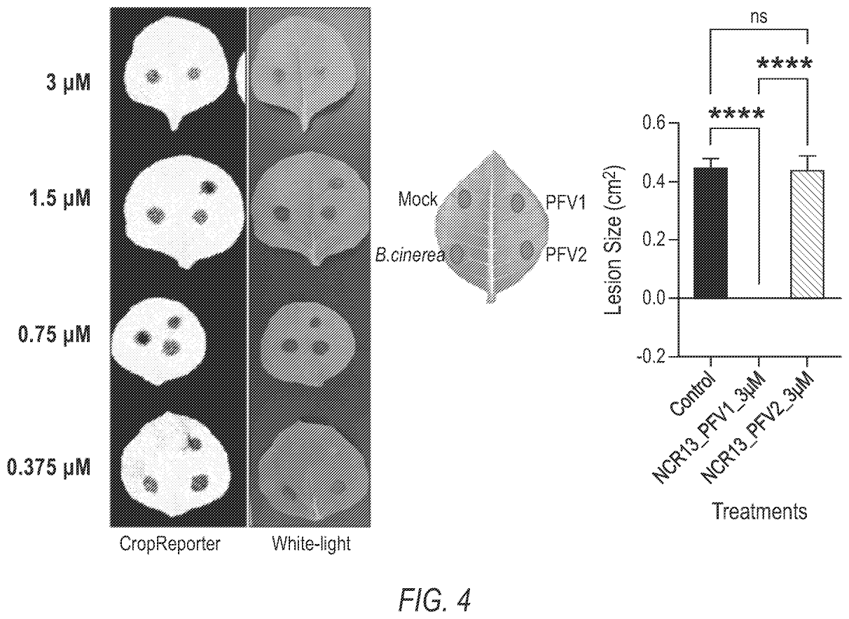

In semi-in-planta studies, detached leaves of N. benthamiana, were used to check the potential of both NCR13_PFV1 and NCR13_PFV2 to reduce gray mold disease symptoms caused by B. cinerea (FIG. 4). NCR13_PFV1 conferred two-fold higher activity against gray mold disease than NCR13_PFV2 in this assay. NCR13_PFV1 was completely inhibitory at 3 μM, whereas NCR13_PFV2 was only partially inhibitory at this concentration.

### Example 7. Semi-In Planta Antifungal Activity of NCR13_PFV1 Against S. sclerotiorum 555

For the semi-in planta antifungal assays, a 1 mm plug was taken from the leading edge of freshly cultured 2-day old fungal colony from PDA media and placed on approximately 3-4 weeks of age detached soybean leaves and pods (6 weeks of age). Then, 40 μL solution of NCR13_PFV1 at various concentrations or water was applied immediately on the plug and were allowed to incubate under high humidity for 2-3 days prior to assessment. The severity of disease lesions on each leaf was assessed using the CropReporter system as described in Li et al. Mol Plant Microbe Interact. 2019; 32:1649-1664. doi: 10.1094/MPMI-08-19-0224-R. Epub 2019 Oct. 28. PMID: 31425003. In addition, high-resolution fluorescence images were taken using CropReporter (PhenoVation, Wageningen, Netherlands).

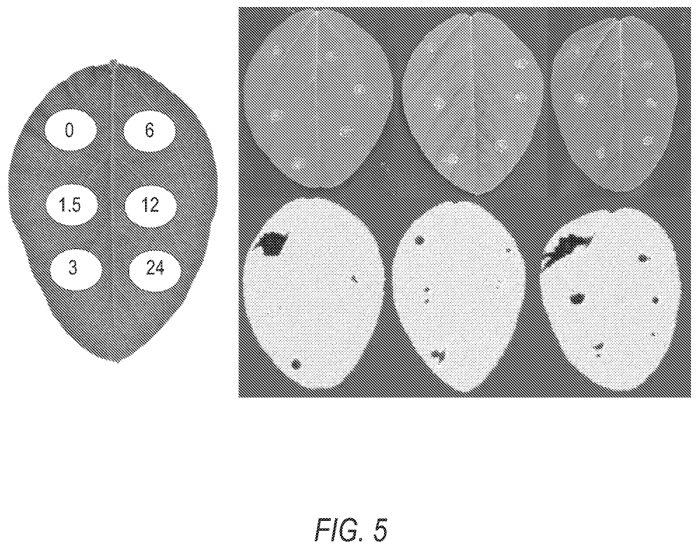

At 2 dpi, leaf parts treated with no peptide showed large lesions. The external application of NCR13_PFV1 to detached leaves significantly decreased lesion sizes with complete inhibition of growth or decay at concentrations between 3 and 12 μM (FIG. 5). The results show that S. sclerotiorum 555 lesion areas were progressively reduced in size with increasing concentrations of NCR13_PFV1.

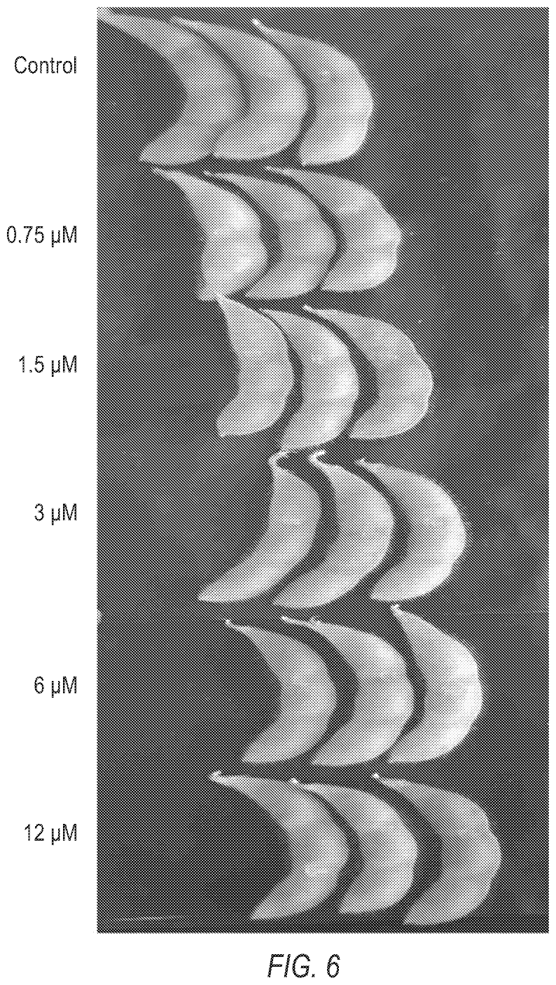

Detached soybean pods from 6-week-old plants were infected with 1 mm plug of S. sclerotiorum 555 followed by the application of a drop of NCR13_PFV1 on the plug. After 3 dpi treated pods exhibited no lesion or decay development compared to the control (water treated). We observed that NCR13_PFV1 confers fungicidal activity with 100% protection against S. sclerotiorum 555 at 3 dpi (FIG. 6).

The results indicate that topical application of NCR13_PFV1 to detached Williams 82 soybean leaves and pods stems significantly decreased lesion sizes with complete inhibition of growth or decay at concentrations between 3 and 12 μM.

### Example 8. Antifungal Activity of NCR13_PFV1 and NCR13_PFV2 Against Botrytis cinerea Infection in Tomato Fruits

Antifungal activity assays were performed on commercially available tomato fruits. A total of 36 tomato fruits (12 fruits per treatment) were washed with 0.01% bleach and rinsed three times with sterile distilled water. Carborundum Powder (Fisher Chemical, USA) was gently rubbed on the fruit surface to create microinjuries. The fruits were inoculated with 20 μl B. cinerea conidia (1×10⁵ conidia ml-1) via drop inoculation. Conidia of B. cinerea were prepared as described above. Fruits were incubated in a Ziploc weather shield box containing moist paper towels to maintain high humidity after drop inoculation. 20 μl of peptide solution (0.75 μM of NCR13_PFV1 or NCR13_PFV2) was applied to the drop inoculated conidial suspension at 12 hours post-inoculation (hpi) and incubated for 5 days. The fruits were observed for fungal growth daily.

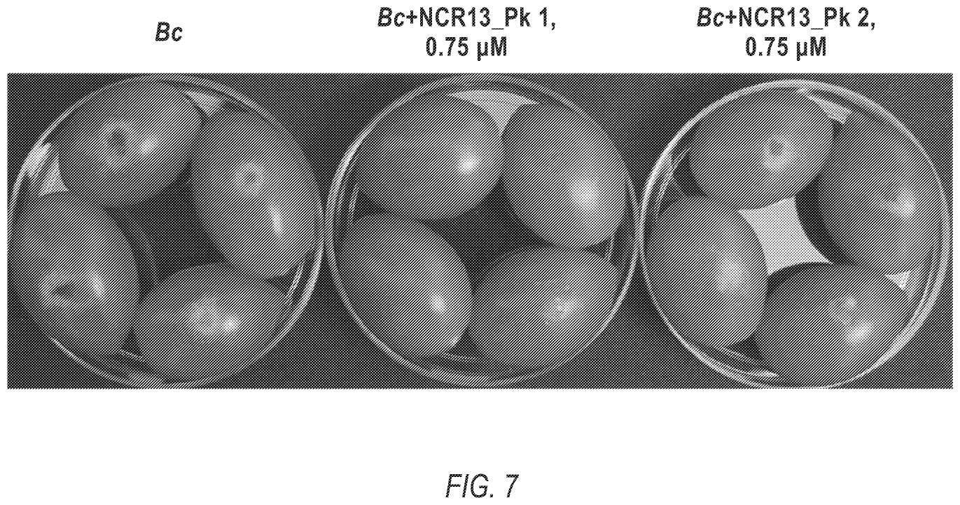

This tomato fruit infection assay revealed that NCR13_PFV1 was superior to NCR13_PFV2 in limiting the growth of B. cinerea at 0.75 μM concentrations (2×MIC) (FIG. 7). At 3 days post-infection (dpi), both the control (B. cinerea conidia without peptide) and NCR13_PFV2 treated fruits had visible fungal growth, whereas NCR-13_PFV1 treated fruits had no visible fungal growth. Even at 5 dpi, NCR13_PFV1 was effective in controlling B. cinerea fungal growth in tomato fruits.

### Example 9. Biological Sequences and Associated SEQ ID NO

Table 4 below lists non-limiting examples of amino acid sequences referred to throughout the disclosure.

TABLE 4
Biological sequences

| SEQ | | | |
|---|---|---|---|
| ID | | | |
| NO. | DESCRIPTION | SEQUENCE | REMARKS |
| 1 | CaNCR13 | TKPCQSDKDCKKFACRKPKVPKCIN | Mature NCR13 |
| | | GFCKCVR | peptide |
| 2 | CaNCR13-Ala | ATKPCQSDKDCKKFACRKPKVPKCI | Mature NCR13-Ala |
| | | NGFCKCVR | peptide |
| 3 | CaNCR07 | KKMPCKRRRDCKTYPCPHPKVRDCV | |
| | | KGYCKCVVR | |
| 4 | CaNCR15 | TKQPCKSRKHCKTYRCPTPKVPNCV | |
| | | NGFCKCVR | |
| 5 | CaNCR14 | SRDCKTYPCPPSKVKDCIKGYCKCV | |
| | | R | |
| 6 | Synthetic gene | CTCGAGAAAAGAGCTACTAAGCCAT | |
| | encoding NCR - | GTCAATCTGATAAGGATTGTAAGAA | |
| | Ala (Alanine | ATTTGCTTGTAGAAAGCCAAAAGTT | |
| | codon in bold) | CCTAAGTGTATTAACGGTTTCTGTA | |
| | | AATGTGTTAGATAGTAATCTAGA | |
| 7 | Consensus of | X₁KX₂PCX₃X₄X₅X₆CKX₇X₈X₉CX₁₀X₁₁ | X₁ is Kor T; X₂ is |
| | CaNCR07, | X₁₂KVX₁₃X₁₄CX₁₅X₁₆GX₁₇CKCVX₁₈R | absent, M, or Q; X₃ |
| | CaNCR13, | | is K or Q; X₄ is R |
| | CaNCR14, and | | or S; X₅ is R or D; |
| | CaNCR15 | | X₆ is R, K, or D; X₇ |
| | | | is T or K; X₈ is Y |
| | | | or F; X₉ is P, A, or |
| | | | R; X₁₀ is P or R; |
| | | | X₁₁ is H, K, P, or T; |
| | | | X₁₂ is P or S; X₁₃ is |
| | | | R, K, or P; X₁₄ is D, |
| | | | K, or N; X₁₅ is I or |
| | | | V; X₁₆ is K or N; |
| | | | X₁₇ is F or Y; X₁₈ is |
| | | | V or absent |
| 8 | CaNCR07 Core | GYCKCVVR | |
| 9 | CaNCR13 Core | GFCKCVR | |
| 10 | CaNCR14 Core | GYCKCVR | |
| 11 | Core Consensus | GX₁CKCVX₂R | wherein X₁ is F or |
| | (with CaNCR14 | | Y and X₂ is absent |
| | core) | | or V |
| 12 | C-terminal 15 | KVX₁X₂CX₃X₄GX₅CKCVX₆R | X₁ is R, K, or P; X₂ |
| | amino acids of | | is D, K, or N; X₃ is |
| | Consensus | | I or V; X₄ is K or |
| | | | N; X₅ is F or Y; X₆ |
| | | | is V or absent |
| 13 | C-terminal 14 | KVPKCINGFCKCVR | |
| | amino acids of | | |
| | CaNCR13 | | |
| 14 | C-terminal 15 | KVRDCVKGYCKCVVR | |
| | amino acids of | | |
| | CaNCR7 | | |
| 15 | C-terminal 14 | KVPNCVNGFCKCVR | |
| | amino acids of | | |
| | CaNCR15 | | |
| 16 | C-terminal 14 | KVKDCIKGYCKCVR | |
| | amino acids of | | |
| | CaNCR14 | | |
| 17 | KEX2 cleavage | KREAEA | |
| | site | | |
| 18 | CaNCR13 C4S, | TKPSQSDKDC₂KKFAC₃RKPKVPKSI | |
| | C23S | NGFC₅KC₆VR | |
| 19 | CaNCR13 C10S, | TKPC₁QSDKDSKKFAC₃RKPKVPKC₄ | |
| | C28S | INGFSKC₆VR | |
| 20 | Consensus of | X₁KX₂PZX₃X₄X₅X₆CKX₇X₈X₉CX₁₀X₁₁ | X₁ is Kor T; X₂ is |
| | CaNCR07, | X₁₂KVX₁₃X₁₄ZX₁₅X₁₆GX₁₇CKCVX₁₈R | absent, M, or Q; X₃ |
| | CaNCR13, | | is K or Q; X₄ is R |
| | CaNCR14, and | | or S; X₅ is R or D; |
| | CaNCR15 with | | X₆ is R, K, or D; X₇ |
| | conserved C₁ | | is T or K; X₈ is Y |
| | and C₄ cysteine | | or F; X₉ is P, A, or |
| | substitutions | | R; X₁₀ is P or R; |
| | | | X₁₁ is H, K, P, or T; |
| | | | X₁₂ is P or S; X₁₃ is |
| | | | R, K, or P; X₁₄ is D, |
| | | | K, or N; X₁₅ is I or |
| | | | V; X₁₆ is K or N; |
| | | | X₁₇ is F or Y; X₁₈ is |
| | | | V or absent; Z is G, |
| | | | S, T, Y, N, or Q |
| 21 | Consensus of | X₁KX₂PCX₃X₄X₅X₆ZKX₇X₈X₉CX₁₀X₁₁ | X₁ is K or T; X₂ is |
| | CaNCR07, | X₁₂KVX₁₃X₁₄CX₁₅X₁₆GX₁₇ZKCVX₁₈R | absent, M, or Q; X₃ |
| | CaNCR13, | | is K or Q; X₄ is R |
| | CaNCR14, and | | or S; X₅ is R or D; |
| | CaNCR15 with | | X₆ is R, K, or D; X₇ |
| | conserved C₂ | | is T or K; X₈ is Y |
| | and C₅ cysteine | | or F; X₉ is P, A, or |
| | substitutions | | R; X₁₀ is P or R; |
| | | | X₁₁ is H, K, P, or T; |
| | | | X₁₂ is P or S; X₁₃ is |
| | | | R, K, or P; X₁₄ is D, |
| | | | K, or N; X₁₅ is I or |
| | | | V; X₁₆ is K or N; Z |
| | | | is G, S, T, Y, N, or |
| | | | Q X₁₇ is F or Y; X₁₈ |
| | | | is V or absent; Z is |

The breadth and scope of the present disclosure should not be limited by any of the above-described examples.

What is claimed is:

1. A composition comprising;
(i) an NCR13 peptide folding variant 1 (NCR PFV1), wherein NCR PFV1 comprises an amino acid sequence having at least 60%, 70%, 80%, 85%, 90%, 92%, 95%, 97%, 98%, 99%, or 100% sequence identity to SEQ ID NO: 1, wherein cysteine residues corresponding to the C4, C10, C15, C23, C28, and C30 cysteine residues of SEQ ID NO: 1 are conserved, wherein said cysteine residues of NCR PFV1 form disulfide bonds between cysteine residues corresponding to C4 to C23, C10 to C28, and C15 to C30, and wherein the composition is substantially free of an NCR13 peptide folding variant 2 (NCR PFV2) comprising the amino acid sequence of NCR PFV1, wherein said cysteine residues of NCR PFV2 form disulfide bonds between cysteine residues corresponding to C4 to C10, C15 to C30, and C23 to C28; and
(ii) an agriculturally, pharmaceutically, or veterinary practicable carrier, diluent, or excipient.

2. The composition of claim 1, wherein said NCR PFV1 and NCR PFV2 peptide comprise: (i) one or more substitutions of a hydrophobic, basic, and/or acidic amino acid residues of SEQ ID NO: 1 with hydrophobic, basic, and/or acidic amino acid residues, respectively; (ii) an amino acid sequence having at least 85%, 90%, 92%, 95%, 97%, 98%, or 99% sequence identity across the entire length of SEQ ID NO: 1 or SEQ ID NO: 2.

3. The composition of claim 1, wherein less than 5%, 2%, 1%, or 0.5% of the total NCR peptide in the composition by weight is NCR PFV2 or wherein at least about 80%, 90%, 95%, 98%, 99%, or 99.5% of the total NCR peptide in the composition by weight is NCR PFV1.

4. The composition of claim 1, wherein less than 5%, 2%, 1%, or 0.5% of the combined total of NCR PFV1 and NCR PFV2 by weight is NCR PFV2.

5. A method of making the composition of any one of claims 1 to 4 comprising:
(i) separating a fraction comprising the NCR13 peptide folding variant 1 (NCR PFV1) from one or more fractions comprising the NCR13 peptide folding variant 2 (NCR PFV2) or NCR PFV2 peptide fragments from a mixture comprising NCR PFV1 and NCR PFV2 or NCR PFV2 peptide fragments; and
(ii) combining the fraction comprising the NCR13 peptide folding variant 1 (NCR PFV1) or a further purified preparation therefrom with an agriculturally, pharmaceutically, or veterinary practicable carrier, diluent, or excipient, thereby making the composition.

6. The method of claim 5, wherein the method further comprises obtaining the mixture by culturing microorganisms comprising a recombinant polynucleotide comprising a promoter which is operably linked to a polynucleotide encoding a signal peptide which is operably linked to a polypeptide comprising SEQ ID NO: 1 or a variant thereof in media and separating the microorganisms from a fermentation broth comprising the mixture, optionally wherein the microorganisms comprise Candida, Kluveromyces, Hansenula, Pichia, Saccharomyces, Schizosaccharomyces, or Yarrowia, Myceliophthora, or Escherichia microorganisms

7. The method of claim 5, wherein the method further comprises obtaining the mixture by disrupting a culture of microorganisms comprising a recombinant polynucleotide comprising a promoter which is operably linked to a polynucleotide encoding a polypeptide comprising SEQ ID NO: 1 or a variant thereof to obtain a cell lysate and separating insoluble debris in the cell lysate from an aqueous fraction comprising the mixture, optionally wherein the microorganisms comprise Candida, Kluveromyces, Hansenula, Pichia, Saccharomyces, Schizosaccharomyces, or Yarrowia, Myceliophthora, or Escherichia microorganisms.

8. The method of claim 5, wherein the separating further comprises treating the mixture comprising NCR PFV1 and NCR PFV2 with a protease at a concentration sufficient to produce the NCR PFV2 peptide fragments but insufficient to produce NCR PFV1 peptide fragments.

9. The method of claim 8, wherein the protease comprises a trypsin family serine protease which cleaves NCR PFV2 at the carboxy terminus of an NCR PFV2 arginine or lysine residue under non-denaturing conditions.

10. The method of claim 9, wherein the trypsin family serine protease is a recombinant bovine, porcine, human, or microbial trypsin, optionally wherein the microbial trypsin is a Streptomycete trypsin or variant thereof.

11. The method of claim 8, wherein the NCR PFV1 is separated from the NCR PFV2 peptide fragments by size-exclusion chromatography.

12. The method of claim 5, wherein the separating is effected by chromatography.

13. A method for preventing or reducing crop damage and/or post-harvest loss by a plant pathogenic microbe comprising the step of contacting a plant, a plant seed, pre- or post-harvest grain, pre- or post-harvest fruit, pre- or post-harvest vegetable, or other part of said plant with an effective amount of the composition of any one of claims 1 to 4.

14. The method of claim 13, wherein the plant pathogenic microbe is a Fusarium sp., Alternaria sp., Verticillium sp., Phytophthora sp., Colletotrichum sp., Botrytis sp., Cercospora sp., Phakopsora sp. Rhizoctonia sp., Sclerotinia sp., Pythium sp., Phoma sp., Leptosphaeria sp., Gaeumannomyces sp., Puccinia sp. Septoria sp., Penicillium sp., Lasiodiplodia sp., Phomopsis sp., Mycosphaerella sp., Golovinomyces sp., Erisyphe sp., Albugo sp., Setosphaeria sp., Cochliobolus sp., Helminthosporium sp., Diplodia sp. or Stenocarpella sp.

15. The method of claim 13, wherein the plant, a plant seed, pre- or post-harvest grain, pre- or post-harvest fruit, or pre- or post-harvest vegetable is a cereal crop, legume crop, root or tuber crop, oil crop, fruit crop, vegetable crop, nut crop, forage or turf grass crop, forage legume, drug crop, spice or flavoring crop, fiber crop, or biofuel crop plant, a plant seed, pre- or post-harvest grain, pre- or post-harvest fruit, or pre- or post-harvest vegetable.

16. The method of claim 15, the plant, a plant seed, pre- or post-harvest grain, pre- or post-harvest fruit, or pre- or post-harvest vegetable is a wheat, rice, corn, barley, oat, sorghum, rye, millet, peanut, chickpea, pigeon pea, lentil, kidney bean, soybean, lima bean, potato, sweet potato, cassava, canola, peanut, palm, coconut, safflower, cotton, sunflower, flax, olive, sugarcane, sugarbeet, banana, orange, apple, pear, breadfruit, pineapple, strawberry, grape, cherry, coffee, coco, tomato, lettuce, carrot, melon, asparagus, Brassica sp., cashew, walnut, pistachio, almond, alfalfa, clover, castor, Camelina, pennycress, switchgrass, Miscanthus, or Jatropha plant, a plant seed, pre- or post-harvest grain, pre- or post-harvest fruit, or pre- or post-harvest vegetable.

17. A medical device comprising the composition of any one of claims 1 to 4, wherein the device comprises at least one surface that is topically coated and/or impregnated with the composition.

18. The medical device of claim 17, wherein said device is a stent, a catheter, a contact lens, a condom, a patch, or a diaphragm.

19. A method for treating, preventing, or inhibiting a microbial infection in a subject in need thereof comprising administering to said subject an effective amount of the composition of any one of claims 1 to 4.

20. The method of claim 19, wherein said administration comprises topical, enteral, parenteral, and/or intravenous introduction of the composition.

21. The method of claim 19, wherein the subject is a human, livestock, poultry, fish, or a companion animal.

22. The method of claim 19, wherein the microbial infection is of a mucosal membrane, eye, skin, and/or a nail and the composition is applied to the mucosal membrane, eye, skin, and/or nail.

23. The method of claim 19, wherein the microbial infection is by a dermatophyte, and wherein the dermatophyte is optionally selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.

24. The method of claim 19, wherein the microbial infection is by an Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, or Blastomyces species.

25. The method of claim 19, wherein the microbial infection is by a Candida species and wherein the Candida species is Candida albicans (C. albicans), C. auris, C. glabrata, C parasilosis, C. tropicalis, or C. krusei.

26. The composition of any one of claim 1 to 4 for use in a method of treating, preventing, or inhibiting microbial infection in a subject in need thereof.

27. The composition of claim 26, wherein the subject is a human, livestock, poultry, fish, or a companion animal.

28. A plant part that is at least partly coated with the composition of any one of claims 1 to 4.

29. The plant part of claim 28, wherein the part is a seed or a pre- or post-harvest grain.

30. The plant part of claim 28, wherein the plant part is a pre- or post-harvest fruit, vegetable, or flower.

31. The plant part of claim 28, wherein the plant part is a cereal crop, legume crop, root or tuber crop, oil crop, fruit crop, vegetable crop, nut crop, forage or turf grass crop, forage legume, drug crop, spice or flavoring crop, fiber crop, or biofuel crop plant part.

32. The plant part of claim 29, wherein the plant part is a wheat, rice, corn, barley, oat, sorghum, rye, millet, peanut, chickpea, pigeon pea, lentil, kidney bean, soybean, lima bean, potato, sweet potato, cassava, canola, peanut, palm, coconut, safflower, cotton, sunflower, flax, olive, sugarcane, sugarbeet, banana, orange, apple, pear, breadfruit, pineapple, strawberry, grape, cherry, coffee, coco, tomato, lettuce, carrot, melon, asparagus, Brassica sp., cashew, walnut, pistachio, almond, alfalfa, clover, castor, Camelina, pennycress, switchgrass, Miscanthus, or Jatropha plant part.
